SEQUENCE LISTING (9 sequences)

SEQ ID NO: 1 - DNA, 1317 bp, Homo sapiens
atgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctga

SEQ ID NO: 2 - protein, 438 aa, Homo sapiens
MGGCAGSRRRFSDSEGEETVPEPRLPLLDHQGAHWKNAVGFWLLGLCNNFSYVVMLSAAHDILSHKRTSGNQSHVDPGPTPIPHNSSSRFDCNSVSTAAVLLADILPTLVIKLLAPLGLHLLPYSPRVLVSGICAAGSFVLVAFSHSVGTSLCGVVFASISSGLGEVTFLSLTAFYPRAVISWWSSGTGGAGLLGALSYLGLTQAGLSPQQTLLSMLGIPALLLASYFLLLTSPEAQDPGGEEEAESAARQPLIRTEAPESKPGSSSSLSLRERWTVFKGLLWYIVPLVVVYFAEYFINQGLFELLFFWNTSLSHAQQYRWYQMLYQAGVFASRSSLRCCRIRFTWALALLQCLNLVFLLADVWFGFLPSIYLVFLIILYEGLLGGAAYVNTFHNIALETSDEHREFAMAATCISDTLGISLSGLLALPLHDFLCQLS

SEQ ID NO: 3 - DNA, 936 bp, Homo sapiens
atggaggcgacgcggaggcggcagcacctgggagcgacgggcggcccaggcgcgcagctgggcgcctccttcctgcaggccaggcatggctctgtgagcgctgatgaggctgcccgcacggctcccttccacctcgacctctggttctacttcacactgcagaactgggttctggactttgggcgtcccattgccatgctggtattccctctcgagtggtttccactcaacaagcccagtgttggggactacttccacatggcctacaacgtcatcacgccctttctcttgctcaagctcatcgagcggtccccccgcaccctgccacgctccatcacgtacgtgagcatcatcatcttcatcatgggtgccagcatccacctggtgggtgactctgtcaaccaccgcctgctcttcagtggctaccagcaccacctgtctgtccgtgagaaccccatcatcaagaatctcaagccggagacgctgatcgactcctttgagctgctctactattatgatgagtacctgggtcactgcatgtggtacatccccttcttcctcatcctcttcatgtacttcagcggctgctttactgcctctaaagctgagagcttgattccagggcctgccctgctcctggtggcacccagtggcctgtactactggtacctggtcaccgagggccagatcttcatcctcttcatcttcaccttcttcgccatgctggccctcgtcctgcaccagaagcgcaagcgcctcttcctggacagcaacggcctcttcctcttctcctccttcgcactgaccctcttgcttgtggcgctctgggtcgcctggctgtggaatgaccctgttctcaggaagaagtacccgggtgtcatctacgtccctgagccctgggctttctacacccttcacgtcagcagtcggcactga

SEQ ID NO: 4 - protein, 311 aa, Homo sapiens
MEATRRRQHLGATGGPGAQLGASFLQARHGSVSADEAARTAPFHLDLWFYFTLQNWVLDFGRPIAMLVFPLEWFPLNKPSVGDYFHMAYNVITPFLLLKLIERSPRTLPRSITYVSIIIFIMGASIHLVGDSVNHRLLFSGYQHHLSVRENPIIKNLKPETLIDSFELLYYYDEYLGHCMWYIPFFLILFMYFSGCFTASKAESLIPGPALLLVAPSGLYYWYLVTEGQIFILFIFTFFAMLALVLHQKRKRLFLDSNGLFLFSSFALTLLLVALWVAWLWNDPVLRKKYPGVIYVPEPWAFYTLHVSSRH

SEQ ID NO: 5 - DNA, 861 bp, Homo sapiens
atgaatcctgcgagcgatgggggcacatcagagagcatttttgacctggactatgcatcctgggggatccgctccacgctgatggtcgctggctttgtcttctacttgggcgtctttgtggtctgccaccagctgtcctcttccctgaatgccacttaccgttctttggtggccagagagaaggtcttctgggacctggcggccacgcgtgcagtctttggtgttcagagcacagccgcaggcctgtgggctctgctgggggaccctgtgctgcatgccgacaaggcgcgtggccagcagaactggtgctggtttcacatcacgacagcaacgggattcttttgctttgaaaatgttgcagtccacctgtccaacttgatcttccggacatttgacttgtttctggttatccaccatctctttgcctttcttgggtttcttggctgcttggtcaatctccaagctggccactatctagctatgaccacgttgctcctggagatgagcacgccctttacctgcgtttcctggatgctcttaaaggcgggctggtccgagtctctgttttggaagctcaaccagtggctgatgattcacatgtttcactgccgcatggttctaacctaccacatgtggtgggtgtgtttctggcactgggacggcctggtcagcagcctgtatctgcctcatttgacactgttccttgtcggactggctctgcttacgctaatcattaatccatattggacccataagaagactcagcagcttctcaatccggtggactggaacttcgcacagccagaagccaagagcaggccagaaggcaacgggcagctgctgcggaagaagaggccatag

SEQ ID NO: 6 - protein, 286 aa, Homo sapiens
MNPASDGGTSESIFDLDYASWGIRSTLMVAGFVFYLGVFVVCHQLSSSLNATYRSLVAREKVFWDLAATRAVFGVQSTAAGLWALLGDPVLHADKARGQQNWCWFHITTATGFFCFENVAVHLSNLIFRTFDLFLVIHHLFAFLGFLGCLVNLQAGHYLAMTTLLLEMSTPFTCVSWMLLKAGWSESLFWKLNQWLMIHMFHCRMVLTYHMWWVCFWHWDGLVSSLYLPHLTLFLVGLALLTLIINPYWTHKKTQQLLNPVDWNFAQPEAKSRPEGNGQLLRKKRP

SEQ ID NO: 7 - DNA, 270 bp, Homo sapiens
ccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggag

SEQ ID NO: 8 - DNA, 280 bp, Homo sapiens
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctac

SEQ ID NO: 9 - DNA, 546 bp, Homo sapiens
gaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaa

(19) United States
(12) Patent Application Publication
(10) Pub. No.: US 2022/0226507 A1
(43) Pub. Date: Jul. 21, 2022

(54) OPTIMIZED GENE THERAPY TARGETING RETINAL CELLS

(71) Applicants: RESEARCH INSTITUTE AT NATIONWIDE CHILDREN'S HOSPITAL, Columbus, OH (US); SANFORD RESEARCH, Sioux Falls, SD (US)

(72) Inventors: Kathrin Christine Meyer, Columbus, OH (US); Shibi Likhite, Columbus, OH (US); Brian K. Kaspar, Westerville, OH (US); Jill M. Weimer, Sioux Falls, SD (US)

(21) Appl. No.: 17/611,982

(22) PCT Filed: Apr. 15, 2020
(86) PCT No.: PCT/US20/28352

§ 371 Date: Nov. 17, 2021

Related U.S. Application Data
(60) Provisional application No. 62/849,794, filed on May 17, 2019.

(51) Int. Cl. A61K 48/00; C12N 15/86; A61P 27/02; C12N 9/24; A61K 9/00

(52) U.S. Cl. CPC A61K 48/0075; C12N 15/86; A61P 27/02; C12N 9/2402; C12N 2750/14171; C12Y 302/01129; A61K 48/005; A61K 9/0048; C12N 2750/14143; C12Y 302/01018

(57) ABSTRACT

The present disclosure relates to methods of targeting specific cell types within the retina using optimized gene therapy vectors. In particular, the disclosure provides gene therapy vectors to specifically target retinal cells and methods of treating visual impairment, retinal degeneration and vision-related disorders such as CLN disease.

This application claims priority to U.S. Provisional Application No. 62/849,794 filed on May 17, 2020, which is incorporated by reference herein in its entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “53953_Seqlisting.txt”, which was created on Apr. 14, 2020 and is 15,429 bytes in size. The subject matter of the Sequence Listing is incorporated herein in its entirety by reference.

### FIELD

The present disclosure relates to methods of targeting specific cell types within the retina using optimized gene therapy vectors. In particular, the disclosure provides gene therapy vectors to specifically target retinal cells and methods of treating visual impairment, retinal degeneration and vision-related disorders such as CLN disease.

### BACKGROUND

Ocular administration of gene therapy vectors has many advantages due to the well-defined anatomy of the eye. In particular, the eye's easy accessibility enables rapid and progressive examinations; the relatively enclosed structure and small size of the eye require lower doses of vector for delivery; the blood-retinal barrier prevents the leakage of vectors into systemic circulation, maintaining a relatively immune-privileged environment; and individual or multiple genes primarily or partially involved in particular ocular disorders have been identified.

Ocular administration of gene therapy vectors has shown some promising results. Currently, there are a number of clinical gene therapy trials targeting vision-loss related diseases, and these trials mainly target hereditary retinal disease. For example, clinical trials have investigated Leber congenital amaurosis (LCA), leber's hereditary optic neuropathy and retinitis pigmentosa. To date, AAV vectors, particular AAV2 serotype, have been the most commonly used in ocular gene therapy. See Lee et al., Progress in retinal and eye research 68: 31-53, 2019

Neuronal ceroid lipofuscinoses (NCLs) are a group of severe neurodegenerative disorders, which are collectively referred to as Batten disease. These disorders affect the nervous system and typically cause worsening problems with e.g. movement, vision and thinking ability. The different NCLs are distinguished by their genetic cause. Partial or complete loss of vision often develops in patients who have childhood forms of Batten disease. In particular, in people suffering from Batten disease, lipofuscin accumulates inside cells, including those of the brain and retina. The buildup of lipofuscin damages the photoreceptors in the retina, optic nerve, and area of the brain that processes vision.

Currently, there is a need for improved gene therapy methods that target specific cell types in the retina. Furthermore, there are no therapies that can reverse the symptoms of Batten Disease. Thus, there is a need in the art for treatments for Batten Disease.

### SUMMARY

The disclosure provides optimized gene therapy vectors that target specific cell types in the retina. These optimized gene therapy vectors are useful for delivering a transgene to specific retinal cells. The disclosure provides for methods of treating a vision-related disorder comprising administering the optimized gene therapy vectors using local intravenous (IV) delivery, sub-retinal delivery, intravitreal delivery, intracerebroventricular delivery, intraparenchymal delivery or intrathecal delivery. Gene therapy methods that target specific cells types have advantages for treating vision-loss related diseases.

The disclosure provides for methods of delivering a transgene to a retinal cell in a subject comprising administering to the subject a gene therapy vector encoding the transgene, wherein the gene therapy vector is administered to the subject using local intravenous (IV) delivery, sub-retinal delivery, intravitreal delivery, intracerebroventricular delivery, intraparenchymal delivery or intrathecal delivery. For example, the disclosed methods result in delivering the transgene to all retinal cells including but not limited to bipolar cell, rod photoreceptor cell, cone photoreceptor cell, ganglion cell, Mueller glia cell, microglia cell, horizontal cell and/or amacrine cell.

The disclosure also provides for a composition for delivering a transgene to a retinal cell in a subject, wherein the composition comprises a gene therapy vector encoding the transgene, wherein the composition is formulated for administering the gene therapy vector using local intravenous delivery, sub-retinal delivery, intravitreous delivery or intrathecal delivery.

In another embodiment, the disclosure provides for use of a gene therapy vector for the preparation of a medicament for delivering a transgene to a retinal cell in a subject, wherein the medicament comprises a gene therapy vector encoding the transgene, and wherein the medicament is formulated for administering the gene therapy vector using local intravenous delivery, sub-retinal delivery, intravitreous delivery or intrathecal delivery

The disclosure also provides for methods of treating visual impairment, retinal degeneration or a vision-related disorder in a subject comprising administering to the subject a gene therapy vector encoding a transgene, wherein the gene therapy vector is administered using local intravenous (IV) delivery, sub-retinal delivery, intravitreal delivery, intracerebroventricular delivery, intraparenchymal delivery or intrathecal delivery.

The disclosure also provides for compositions for treating visual impairment or a vision-related disorder in a subject, wherein the composition comprises a gene therapy vector encoding a transgene to the subject, wherein the composition is formulated for administering the gene therapy vector using local intravenous delivery, sub-retinal delivery, intravitreous delivery or intrathecal delivery.

In additional embodiments, the disclosure provides for use of a gene therapy vector the preparation of a medicament for treating visual impairment or a vision-related disorder in a subject, wherein the medicament comprises a gene therapy vector encoding a transgene, wherein the medicament is formulated for administering the gene therapy vector using local intravenous delivery, sub-retinal delivery, intravitreous delivery or intrathecal delivery

For example, the vision-related disorder is Batten disease, congenital cataracts, congenital glaucoma, retinal degeneration, optic atrophy, eye malformations. Strabismus, ocular misalignment, glaucoma, wet age-related macular degeneration, dry age-related macular degeneration, retinitis pigmentosa, choroideremia, Leber congenital amaurosis, Leber's hereditary optic neuropathy, early onset retinal dystrophy, achromatopsia, x-linked retinoschisis, Usher Syndrome 1B, neovascular age-related macular degeneration, Stargardt's macular degeneration, diabetic macular degeneration, or diabetic macular edema. In a particular embodiment, the vision-related disorder is a CLN Batten disease such as CLN1 disease, CLN2 disease, CLN3 disease, CLN4 disease, CLN5 disease, CLN6 disease or CLN8 disease.

The disclosed methods, compositions and uses for delivering any transgene of interest to a retinal cell. The transgene is a polynucleotide sequence that encodes a polypeptide of interest or is a nucleic acid that inhibits, interferes or silences expression of a gene of interest, such as a siRNA or miRNA. Exemplary transgenes are polynucleotides that encode RPE65, RPGR, ORF15, CNGA3, CMH, ND4, PDE6B, ChR2, MERTK, hRS1, hMYOJA, hABCA4, CD59, anti-hVEGF antibody, endostatin-angiostatin, sFLT01, or sFLT-1. Additional exemplary transgenes include siRNA against RTP801, siRNA against VEGFR-1, siRNA against VEGF, or siRNA against ADRB2. In one embodiment, the transgene encodes a CLN polypeptide, such as CLN1, CLN2, CLN3, CLN4, CLN5, CLN6 or CLN8.

The disclosure also provides for methods of treating Batten disease in a subject comprising administering to the subject a gene therapy vector comprising a polynucleotide encoding a CLN polypeptide, wherein the gene therapy vector is administered using local intravenous (IV) delivery, sub-retinal delivery, intravitreal delivery, intracerebroventricular delivery, intraparenchymal delivery or intrathecal delivery.

In other embodiments, the disclosure provides for compositions for treating Batten disease in a subject, wherein the composition comprises a gene therapy vector comprising a polynucleotide encoding a CLN polypeptide, wherein the composition is formulated for administering the gene therapy vector using sub-retinal delivery, intravitreous delivery or intrathecal delivery.

In additional embodiments, the disclosure provides for use of a gene therapy vector for the preparation of a medicament for treating Batten disease in a subject, wherein the medicament comprises a gene therapy vector comprising a polynucleotide encoding a CLN polypeptide, and wherein the medicament is formulated for administering the gene therapy vector using sub-retinal delivery, intravitreous delivery or intrathecal delivery.

The Batten disease treated by any of the methods, compositions or uses of the disclosure is CLN1 disease, CLN2 disease, CLN3 disease, CLN4 disease, CLN5 disease, CLN6 disease or CLN8 disease.

In any of the disclosed methods, compositions or uses, the transgene is a polynucleotide encoding a CLN polypeptide, such as CLN1, CLN2, CLN3, CLN4, CLN5, CLN6 or CLN8. In any of the methods, compositions or uses for treating Batten disease, an effective treatment reduces or slows one or more symptoms of Batten Disease selected from: (a) loss of vision; (b) loss of brain volume; (c) loss of cognitive function; and (d) language delay; as compared to an untreated Batten Disease patient. The symptoms may be evaluated using the Unified Batten Disease Rating Scale (UBDS) or the Hamburg Motor and Language Scale.

In any of the disclosed methods, compositions or uses, the gene therapy vector is AAV1, AAV2, AAV3, AAV4, AAV5, AAV6, AAV7, AAV8, AAV9, AAVRH10, AAVRH74, AAV11, AAV12, AAV13, AAVTT or Anc80, AAV7m8 and their derivatives.

In any of the disclosed methods, compositions or uses, the gene therapy vector comprises a CMV promoter, the p546, or the CB promoter.

In addition, in any of the disclosed methods, compositions or uses, the gene therapy vector is administered using intrathecal delivery, and the method further comprises placing the subject in the Trendelenburg position after administering of the gene therapy vector.

### BRIEF DESCRIPTION OF THE DRAWINGS

FIG. 1 provides the schematic of the vectors tested in this disclosure.

FIGS. 2A-2D demonstrate retinal delivery of the GFP transgene after intrathecal administration.

FIGS. 3A-3E demonstrate retinal delivery of the GFP transgene after intrathecal administration.

FIGS. 4A-4C demonstrate retinal delivery of the GFP transgene after sub-retinal injection.

FIGS. 5A-5C demonstrate retinal delivery of the GFP transgene after sub-retinal delivery.

FIGS. 6A-6B provides a composite of all tissues after sub-retinal delivery at low magnification for all 4 vectors tested using two different co-stains.

FIG. 7 provides data from a 10 year old non-human primate that was intrathecally injected with 1.2e¹⁴ vg of AAV9.CB.GFP. Retinas were counterstained with Sox2, a Mueller glia cell marker. Marked co-localization of GFP and Sox2 was observed in the injected primate.

FIGS. 8A-8B demonstrate transduction of Mueller glia cells in the retina after intravitreal injection. GC is ganglion cell layer, INL is inner neuronal layer and ONL is outer neuronal layer. Sox2 is a Mueller cell specific marker.

FIG. 9A-9B demonstrate transduction of bipolar cells in the retina after intravitreal injection. Otx2 is a bipolar cell specific marker.

### DETAILED DESCRIPTION

Optimization of AAV gene therapy for targeting the eye to treat vision-related disorders, such as Batten Disease, requires specific targeting of different cell types. The disclosure provides experimental data comparing different gene therapy vectors, promoters and routes of administration to determine the optimal gene therapy vector for targeting delivery of a transgene to specific cell types in the retina of mice and non-human primates.

The data focuses on administration of AAV9 and Anc80 vectors, but the disclosure contemplates using any gene therapy vector that comprises a promoter that specifically targets a retinal cell, and these optimized vectors are administered using local intravenous (IV) delivery, sub-retinal delivery, intravitreal delivery, intracerebroventricular delivery, intraparenchymal delivery or intrathecal delivery. For example, the data demonstrated that AAV9 injected directly into the cerebrospinal fluid via intracerebroventricular injection was effective in targeting transgene expression in the bipolar cells of the retina. Thus, intrathecal injections can be used to deliver gene therapy vectors to the eye and specifically for delivering gene therapy vectors to bipolar cells.

### Gene Therapy Vectors

Adeno-associated virus (AAV) is a replication-deficient parvovirus, the single-stranded DNA genome of which is about 4.7 kb in length including two 145 nucleotide inverted terminal repeats (ITRs) and may be used to refer to the virus itself or derivatives thereof. The term covers all subtypes and both naturally occurring and recombinant forms, except where specified otherwise. There are multiple serotypes of AAV. The serotypes of AAV are each associated with a specific clade, the members of which share serologic and functional similarities. Thus, AAVs may also be referred to by the clade. For example, AAV9 sequences are referred to as “clade F” sequences (Gao et al., J. Virol., 78: 6381-6388 (2004). The present disclosure contemplates the use of any sequence within a specific clade, e.g., clade F. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-B1 genome is provided in Choudhury et al., Mol. Ther., 24(7): 1247-1257 (2016). Anc80 is an AAV vector that is of AAV1, AAV2, AAV8 and AAV9. The sequence of Anc80 is provided in Zinn et al., Cell Reports 12: 1056-1068, 2015, Vandenberghe et al, PCT/US2014/060163, both of which are incorporated by reference herein, in their entirety and GenBank Accession Nos. KT235804-KT235812.

Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The native AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. In some instances, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

The term “AAV” as used herein refers to the wild type AAV virus or viral particles. The terms “AAV,” “AAV virus,” and “AAV viral particle” are used interchangeably herein. The term “rAAV” refers to a recombinant AAV virus or recombinant infectious, encapsulated viral particles. The terms “rAAV,” “rAAV virus,” and “rAAV viral particle” are used interchangeably herein.

The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest flanked on the 5′ and 3′ ends by inverted terminal repeat (ITR). In some embodiments, the rAAV genome comprises a “gene cassette.”

The term “scAAV” refers to a rAAV virus or rAAV viral particle comprising a self-complementary genome. The term “ssAAV” refers to a rAAV virus or rAAV viral particle comprising a single-stranded genome.

The rAAV genomes provided herein, in some embodiments, comprise one or more AAV ITRs flanking the transgene polynucleotide sequence. The transgene polynucleotide sequence is operatively linked to transcriptional control elements (including, but not limited to, promoters, enhancers and/or polyadenylation signal sequences) that are functional in target cells to form a gene cassette. Examples of promoters are the CMV promoter, chicken β actin promoter (CB), and the P546 promoter. Additional promoters are contemplated herein including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter.

Additionally provided herein is the CMV promoter sequence comprising the nucleic acid sequence of SEQ ID NO: 8 and promoter sequences that are at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 3 and which exhibit transcription promoting activity. Also provide is the CB promoter sequence comprising the nucleic acid sequence of SEQ ID NO: 7 and promoter sequences that are at least at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO:7 which exhibit transcription promoting activity. In addition, the disclosure provides the P546 promoter sequence comprising the nucleic acid sequence of SEQ ID NO: 9 and promoter sequence that are at least at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 9 which exhibit transcription promoting activity. Other examples of transcription control elements are tissue specific control elements, for example, promoters that allow expression specifically within neurons or specifically within astrocytes. Examples include neuron specific enolase and glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a transgene RNA transcript when expressed in mammalian cells. One example of such an intron is the SV40 intron.

“Packaging” refers to a series of intracellular events that result in the assembly and encapsidation of an AAV particle. The term “production” refers to the process of producing the rAAV (the infectious, encapsulated rAAV particles) by the packing cells.

AAV “rep” and “cap” genes refer to polynucleotide sequences encoding replication and encapsidation proteins, respectively, of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses may encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

The rAAV genomes provided herein lack AAV rep and cap DNA. AAV DNA in the rAAV genomes (e.g., ITRs) contemplated herein may be from any AAV serotype suitable for deriving a recombinant virus including, but not limited to, AAV serotypes Anc80, AAV-1, AAV-2, AAV-3, AAV-4, AAV-5, AAV-6, AAV-7, AAV-8, AAV-9, AAV-10, AAV-11, AAV-12, AAV-13, AAV rh.74 and AAV-B1. As noted above, the nucleotide sequences of the genomes of various AAV serotypes are known in the art. rAAV with capsid mutations, are also contemplated. See, for example, Marsic et al., Molecular Therapy, 22(11): 1900-1909 (2014). Modified capsids herein are also contemplated and include capsids having various post-translational modifications such as glycosylation and deamidation. Deamidation of asparagine or glutamine side chains resulting in conversion of asparagine residues to aspartic acid or isoaspartic acid residues, and conversion of glutamine to glutamic acid or isoglutamic acid is contemplated in rAAV capsids provided herein. See, for example, Giles et al., Molecular Therapy, 26(12): 2848-2862 (2018). Modified capsids herein are also contemplated to comprise targeting sequences directing the rAAV to the affected tissues and organs requiring treatment.

DNA plasmids provided herein comprise rAAV genomes described herein. The DNA plasmids may be transferred to cells permissible for infection with a helper virus of AAV (e.g., adenovirus, E1-deleted adenovirus or herpesvirus) for assembly of the rAAV genome into infectious viral particles with AAV9 capsid proteins. Techniques to produce rAAV, in which an rAAV genome to be packaged, rep and cap genes, and helper virus functions are provided to a cell are standard in the art. Production of rAAV particles requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs. Production of pseudotyped rAAV is disclosed in, for example, WO 01/83692 which is incorporated by reference herein in its entirety. In various embodiments, AAV capsid proteins may be modified to enhance delivery of the recombinant rAAV. Modifications to capsid proteins are generally known in the art. See, for example, US 2005/0053922 and US 2009/0202490, the disclosures of which are incorporated by reference herein in their entirety.

A method of generating a packaging cell is to create a cell line that stably expresses all the necessary components for rAAV production. For example, a plasmid (or multiple plasmids) comprising a rAAV genome lacking AAV rep and cap genes, AAV rep and cap genes separate from the rAAV genome, and a selectable marker, such as a neomycin resistance gene, may be integrated into the genome of a cell. rAAV genomes may be introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line may then be infected with a helper virus such as adenovirus. The advantages of this method are that the cells are selectable and are suitable for large-scale production of rAAV. Other non-limiting examples of suitable methods employ adenovirus or baculovirus rather than plasmids to introduce rAAV genomes and/or rep and cap genes into packaging cells.

General principles of rAAV particle production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV particle production.

Further provided herein are packaging cells that produce infectious rAAV particles. In one embodiment packaging cells may be stably transformed cancer cells such as HeLa cells, 293 cells and PerC.6 cells (a cognate 293 line). In another embodiment, packaging cells may be cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Also provided herein are rAAV (e.g., infectious encapsidated rAAV particles) comprising a rAAV genome of the disclosure. The genomes of the rAAV lack AAV rep and cap DNA, that is, there is no AAV rep or cap DNA between the ITRs of the genomes of the rAAV. The rAAV genome can be a self-complementary (sc) genome. A rAAV with a sc genome is referred to herein as a scAAV. The rAAV genome can be a single-stranded (ss) genome. A rAAV with a single-stranded genome is referred to herein as an ssAAV.

The rAAV may be purified by methods standard in the art such as by column chromatography or cesium chloride gradients. Methods for purifying rAAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

Compositions comprising rAAV are also provided. Compositions comprise a rAAV encoding a CLN6 polypeptide. Compositions may include two or more rAAV encoding different polypeptides of interest. In some embodiments, the rAAV is scAAV or ssAAV.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, copolymers such as poloxamer 188, pluronics (e.g., Pluronic F68) or polyethylene glycol (PEG). Compositions provided herein can comprise a pharmaceutically acceptable aqueous excipient containing a non-ionic, low-osmolar compound such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgI/mL, an osmolality by vapor-pressure osmometry of about 322 mOsm/kg water, an osmolarity of about 273 mOsm/L, an absolute viscosity of about 2.3 cp at 20° C. and about 1.5 cp at 37° C., and a specific gravity of about 1.164 at 37° C. Exemplary compositions comprise about 20 to 40% non-ionic, low-osmolar compound or about 25% to about 35% non-ionic, low-osmolar compound. An exemplary composition comprises scAAV or rAAV viral particles formulated in 20 mM Tris (pH8.0), 1 mM MgCl₂, 200 mM NaCl, 0.001% poloxamer 188 and about 25% to about 35% non-ionic, low-osmolar compound. Another exemplary composition comprises scAAV formulated in and 1X PBS and 0.001% Pluronic F68.

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include about 1×10⁷, 1×10⁸, 1×10⁹ ,5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 1×10¹¹, about 1×10¹², about 1×10¹³, about 1.1×10¹³, about 1.2×10¹³, about 1.3×10¹³, about 1.5×10¹³, about 2×10¹³, about 2.5×10¹³, about 3×10¹³, about 3.5×10¹³, about 4×10¹³, about 4.5×10¹³, about 5×10¹³, about 6×10¹³, about 1×10¹⁴, about 2×10¹⁴, about 3×10¹⁴, about 4×10¹⁴about 5×10¹⁴, about 1×10¹⁵, to about 1×10¹⁶, or more total viral genomes. Dosages of about 1×10⁹ to about 1×10¹⁰, about 5×10⁹ to about 5×10¹⁰, about 1×10₁₀ to about 1×10¹¹, about 1×10¹¹ to about 1×10¹⁵ vg, about 1×10¹² to about 1×10¹⁵ vg, about 1×10¹² to about 1×10¹⁴ vg, about 1×10¹³ to about 6×10¹⁴ vg, and about 6×10¹³ to about 1.0×10¹⁴ vg are also contemplated. One dose exemplified herein is 6×10¹³ vg. Another dose exemplified herein is 1.5×10¹³ vg.

Methods of transducing target retinal cells with rAAV are provided. The retina cells include bipolar cells, rod photoreceptor cells, cone photoreceptor cell, ganglion cell, Mueller glia cells, microglia cells, horizontal cells or amacrine cells.

The term “transduction” is used to refer to the administration/delivery of the CLN6 polynucleotide to a target cell either in vivo or in vitro, via a replication-deficient rAAV of the disclosure resulting in expression of a functional polypeptide by the recipient cell. Transduction of cells with rAAV of the disclosure results in sustained expression of polypeptide or RNA encoded by the rAAV. The present disclosure thus provides methods of administering/delivering to a subject rAAV encoding a transgene encoded polypeptide by an intrathecal, local IV delivery, intracerebroventricular, sub-retinal injection, intravitreous delivery or intraparenchymal delivery, or any combination thereof. Intrathecal delivery refers to delivery into the space under the arachnoid membrane of the brain or spinal cord. In some embodiments, intrathecal administration is via intracisternal administration.

### Transgenes

The disclosed methods of delivery any transgene of interest to a retinal cell. The transgene is a polynucleotide sequence that encodes a polypeptide of interest or is a nucleic acid that inhibits, interferes or silences expression of a gene of interest, such as a siRNA or miRNA.

Exemplary transgenes are polynucleotides that encode RPE65, RPGR, ORF15, CNGA3, CMH, ND4, PDE6B, ChR2, MERTK, hRS1, hMYOJA, hABCA4, CD59, anti-hVEGF antibody, endostatin-angiostatin, sFLT01, or sFLT-1. In one embodiment, the transgene encodes a CLN polypeptide, such as CLN1, CLN2, CLN3, CLN4, CLN5, CLN6 or CLN8. Additional exemplary transgenes include siRNA against RTP801, siRNA against VEGFR-1, siRNA against VEGF, or siRNA against ADRB2.

miRNA that are expressed in the retina are contemplated as transgenes to include in the disclosed optimized gene therapy vectors. Examples of miRNA are provided in Karali et al., Nucleic Acids Res. 2016 Feb. 29; 44(4): 1525-1540, which is incorporated by reference herein.

rAAV genomes provided herein may comprise a polynucleotide encoding a transgene comprising a polynucleotide sequence encoding any one of RPE65, RPGR, ORF15, CNGA3, CMH, ND4, PDE6B, ChR2, MERTK, hRS1, hMYOJA, hABCA4, CD59, PEDF, endostatin-angiostatin genes, sFLT-1, gene encoding an anti-hVEGF antibody. For example, the polypeptide encoded by the transgene include polypeptides comprising an amino acid sequence that is at least: 90%, 91%, 92%, 93%, 94%, 95%, 96%, 97%, 98%, or 99% identical to the amino acid sequence encoded by the transgene sequence.

rAAV genomes provided herein comprise a polynucleotide encoding a CLN polypeptide, such as CLN1, CLN2, CLN3, CLN4, CLN5, CLN6 and CLN8. The polypeptide include polypeptides comprising an amino acid sequence that is at least: 90%, 91%, 92%, 93%, 94%, 95%, 96%, 97%, 98%, or 99% identical to a CLN polypeptide amino acid sequence, and which encodes a polypeptide with CLN activity (e.g., at least one of increasing clearance of lysosomal auto fluorescent storage material, reducing lysosomal accumulation of ATP synthase subunit C, and reducing activation of astrocytes and microglia in a patient when treated as compared to, e.g. the patient prior to treatment).

rAAV genomes provided herein, in some cases, comprise a polynucleotide encoding a CLN polypeptide or a polynucleotide at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence that encodes a polypeptide with CLN activity (e.g., at least one of increasing clearance of lysosomal auto fluorescent storage material, reducing lysosomal accumulation of ATP synthase subunit C, and reducing activation of astrocytes and microglia in a patient when treated as compared to, e.g. the patient prior to treatment).

rAAV genomes provided herein, in some embodiments, comprise a transgene comprising a polynucleotide sequence that encodes a polypeptide with a desired activity and that hybridizes under stringent conditions to any one of nucleic acid sequence of a known transgene of interest, or the complement thereof. In other embodiments, rAAV genomes provided herein comprise a polynucleotide sequence that encodes a polypeptide with CLN activity and that hybridizes under stringent conditions to any one of nucleic acid sequences encoding a CLN polypeptide, or the complement thereof.

The following outlines the disease characteristics of each Batten Disease subtype with emphasis on visual components. The data included in the “Primary Affected Retinal Cell” column was determined based on single cell RNA data compiled from mouse retina. Investigation of this data is still ongoing.

| Batten | | Proposed Gene | Disease | | Primary Affected Retinal |
|---|---|---|---|---|---|
| Disease | Affected Gene | Function | Onset | Vision Loss Onset | Cell |
| CLN1 | PPT1 | Lysosomal enzyme | 6-24 | 2 years | Muller Glia, Ganglion, |
| | | | months | | Horizontal, Amacrine |
| CLN2 | TPP1 | Lysosomal Enzyme | 2-4 years | 4-6 years | Mueller Glia |
| CLN3 | CLN3 | Transmembrane | 4-8 years | Initial Symptom | Mueller Glia |
| | | Protein | | | |
| CLN4 | DNAJC5 | Cytoplasmic Protein | ~30 years | Uncommon | Broad [high] expression in |
| | | | | | all cell types |
| CLN5 | CLN5 | Soluble Lysosomal | 4.5-7 years | 5-11 years | Mueller Glia |
| | | Protein | | | |
| CLN6 | CLN6 | Transmembrane | 18 mos-8 | Initial Symptom | Cone and Rod Bipolars |
| | | Protein | years | | |
| CLN7 | MFSD8 | Transmembrane | 2-7 years | Initial Symptom | Broad [low] expression in |
| | | Protein | | | all cell types |
| CLN8 | CLN8 | Transmembrane | 2-6 years | 2-10 years | Mueller Glia |
| | | Protein | | | |
| CLN9 | Unknown | Unknown | 4-10 years | Initial Symptom | Unknown |
| CLN10 | CTSD | Lysosomal Enzyme | At birth | Not Characterized | Broad [high] expression in |
| | | | | due to early death | all cell types |
| CLN11 | GRN | Secretory Pathway | 15-50 years | Initial symptom | Mueller glia, Microglia |
| | | Protein | | | |
| CLN12 | ATP13A2 | Transmembrane | ~8 years | None | Broad [med-high] |
| | | Protein | | | expression in all cell types |
| CLN13 | CTSF | Lysosomal Enzyme | ~30 years | No vision loss | Broad [high] expression in |
| | | | | | all cell types |
| CLN14 | KCTD7 | Cytoplasmic Protein | 8-24 | Varies; ~4 | Broad [low] expression in |
| | | | months | years | all cell types |

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing include but are not limited to 0.015 M sodium chloride, 0.0015 M sodium citrate at 65-68° C. or 0.015 M sodium chloride, 0.0015M sodium citrate, and 50% formamide at 42° C. See, for example, Sambrook et al., Molecular Cloning: A Laboratory Manual, 2nd Ed., Cold Spring Harbor Laboratory, (Cold Spring Harbor, N.Y. 1989).

### Methods of Administration

Intrathecal administration is exemplified herein. These methods include transducing target cells with one or more rAAV described herein. In some embodiments, the rAAV viral particle comprising a transgene is administered or delivered the eye, brain and/or spinal cord of a patient. In some embodiments, the polynucleotide is delivered to brain. Areas of the brain contemplated for delivery include, but are not limited to, the motor cortex, visual cortex, cerebellum and the brain stem. In some embodiments, the polynucleotide is delivered to the spinal cord. In some embodiments, the polynucleotide is delivered to a lower motor neuron. The polynucleotide may be delivered a retinal cell such as a bipolar cell, rod photoreceptor cell, cone photoreceptor cell, ganglion cell, Mueller glia cell, microglia cell, horizontal cell or amacrine cell.

In some embodiments of methods provided herein, the patient is held in the Trendelenburg position (head down position) after administration of the rAAV (e.g., for about 5, about 10, about 15 or about 20 minutes). For example, the patient may be tilted in the head down position at about 1 degree to about 30 degrees, about 15 to about 30 degrees, about 30 to about 60 degrees, about 60 to about 90 degrees, or about 90 to about 180 degrees).

For sub-retinal administration, a small scleral incision is made, at or posterior to the equator of the eye with a needle, e.g. 30 G needle. Virus or vehicle is delivered sub-retinally via the incision, for example, a fine glass pipette attached by tubing to a Hamilton syringe or via 30 G needle and Hamilton syringe. For example, sub-retinal administration is carried out by a clinically trained surgeon using methods known in the art.

For intracerebroventicular injections, a needle is inserted into the skull and the liquid is injected into the ventricles containing cerebrospinal fluid. For example, intracerebroventicular injections are carried out by a clinically trained surgeon using methods known in the art.

The methods provided herein comprise the step of administering an effective dose, or effective multiple doses, of a composition comprising a rAAV provided herein to a subject (e.g., an animal including, but not limited to, a human patient) in need thereof. If the dose is administered prior to development of the symptoms of the vision-related disorder, the administration is prophylactic. If the dose is administered after the development of symptoms of the vision-related disorder, the administration is therapeutic. An effective dose is a dose that alleviates (eliminates or reduces) at least one symptom associated with the vision-related disorder, that slows or prevents progression of the disorder, that diminishes the extent of disorder, that results in remission (partial or total) of disorder, and/or that prolongs survival and/or vision. In comparison to the subject before treatment or in comparison to an untreated subject, methods provided herein result in stabilization, reduced progression of vision loss or retinal degeneration, or improvement in vision or macular degeneration.

When the vision-related disorder is CLN Batten disease, comparison to the subject before treatment or in comparison to an untreated subject, methods provided herein result in stabilization, reduced progression, or improvement in one or more of the scales that are used to evaluate progression and/or improvement in CLN Batten-disease, e.g. the Unified Batten Disease Rating System (UBDRS) or the Hamburg Motor and Language Scale. The UBDRS assessment scales (as described in Marshall et al., Neurology. 2005 65(2):275-279) [including the UBDRS physical one or more of the scales that are used to evaluate progression and/or improvement in CLN Batten-disease, e.g. the Unified Batten Disease Rating System (UBDRS) or the Hamburg Motor and Language Scale. The UBDRS assessment scales (as described in Marshall et al., Neurology. 2005 65(2):275-279) [including the UBDRS physical assessment scale, the UBDRS seizure assessment scale, the UBDRS behavioral assessment scale, the UBDRS capability assessment scale, the UBDRS sequence of symptom onset, and the UBDRS Clinical Global Impressions (CGI)]; the Pediatric Quality of Life Scale (PEDSQOL) scale, motor function, language function, cognitive function, and survival. In comparison to the subject before treatment or in comparison to an untreated subject, methods provided herein may result in one or more of the following: reduced or slowed lysosomal accumulation of autofluorescent storage material, reduced or slowed lysosomal accumulation of ATP Synthase Subunit C, reduced or slowed glial activation (astrocytes and/or microglia) activation; reduced or slowed astrocytosis, and showed a reduction or delay in brain volume loss measured by MRI.

### EXAMPLES

While the following examples describe specific embodiments, it is understood that variations and modifications will occur to those skilled in the art. Accordingly, only such limitations as appear in the claims should be placed on the invention.

### Example 1

Production of scAAV9.GFP and Anc80.GFP

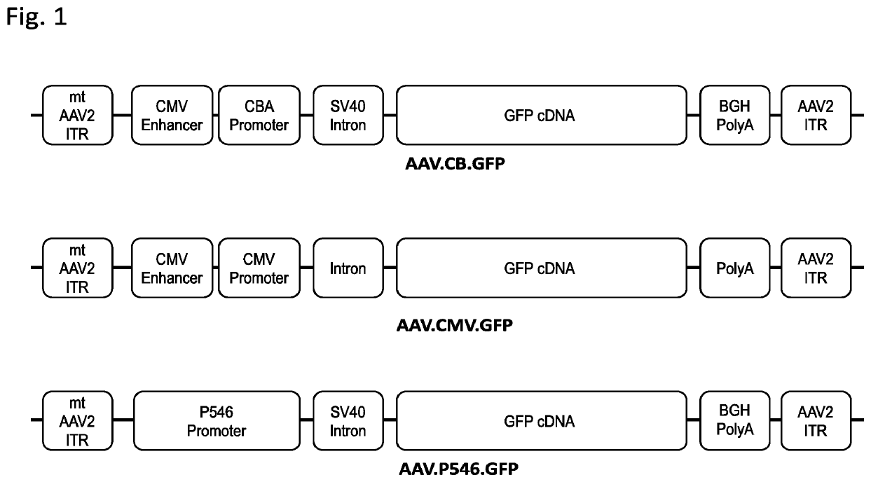

A human GFP cDNA clone was obtained from Origene, Rockville, Md. GFP cDNA was further subcloned into a self complementary AAV9 genome or an Anc80 genome under the hybrid chicken β-Actin promoter (CB), the CMV enhancer-promoter, or the P546 promoter and tested in vitro and in vivo. A schematic of the plasmid constructs showing the GFP cDNA inserted between AAV2 ITRs is provided in FIG. 1. The plasmid construct also included one or more of the CB promoter, an intron such as the simian virus 40 (SV40) chimeric intron and a Bovine Growth Hormone (BGH) polyadenylation signal (BGH PolyA). The constructs in FIG. 1 were packaged into either AAV9 genome or the Anc80 genome (referred to collectively as “AAV”).

### Example 2

Transduction of Mouse Retina After ICV Delivery

scAAV9.CB.GFP was administered to mice via one intracerebroventricular (ICV) injection within Day 0 to Day 2 after birth and expression was monitored at various time points over a course of two months. The mice were injected with an 5e10 vg of scAAV9.CB.GFP. The scAAV9.CB.GFP was formulated in 1x PBS and 0.001% Pluronic F68 (denoted as PBS/F68).

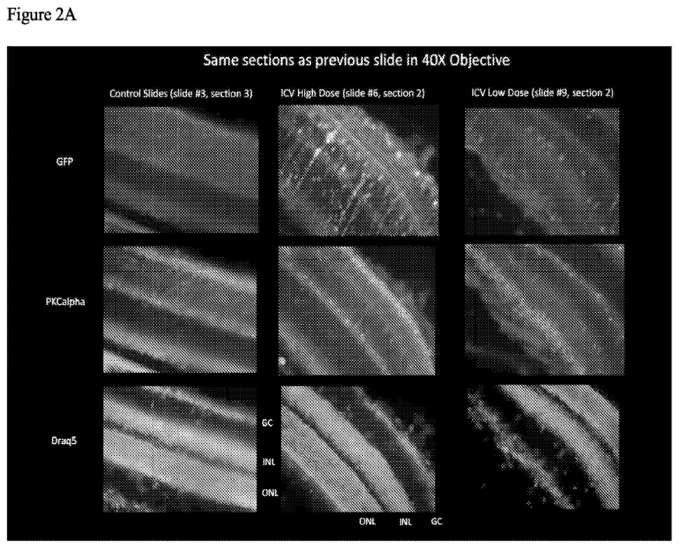

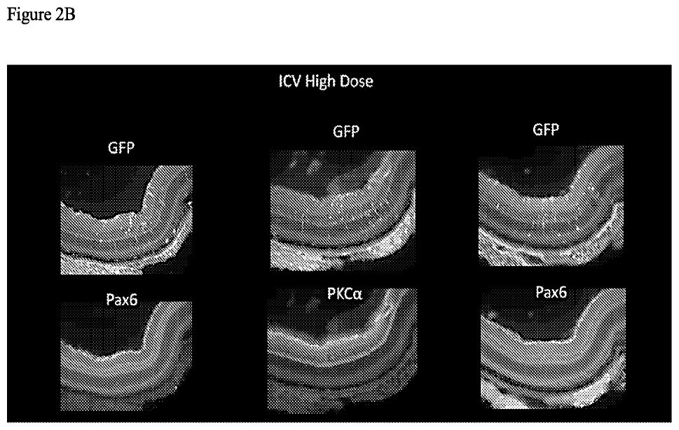

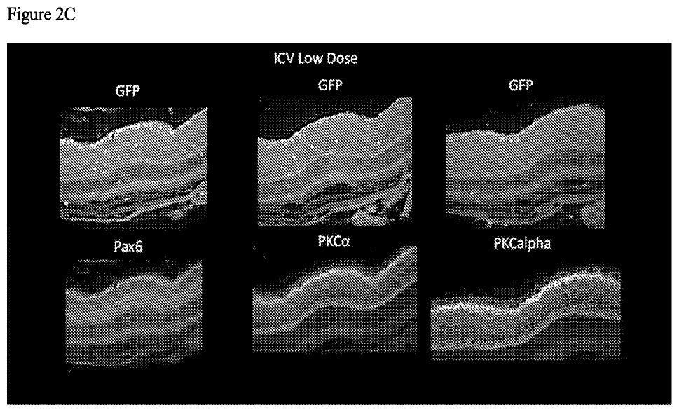

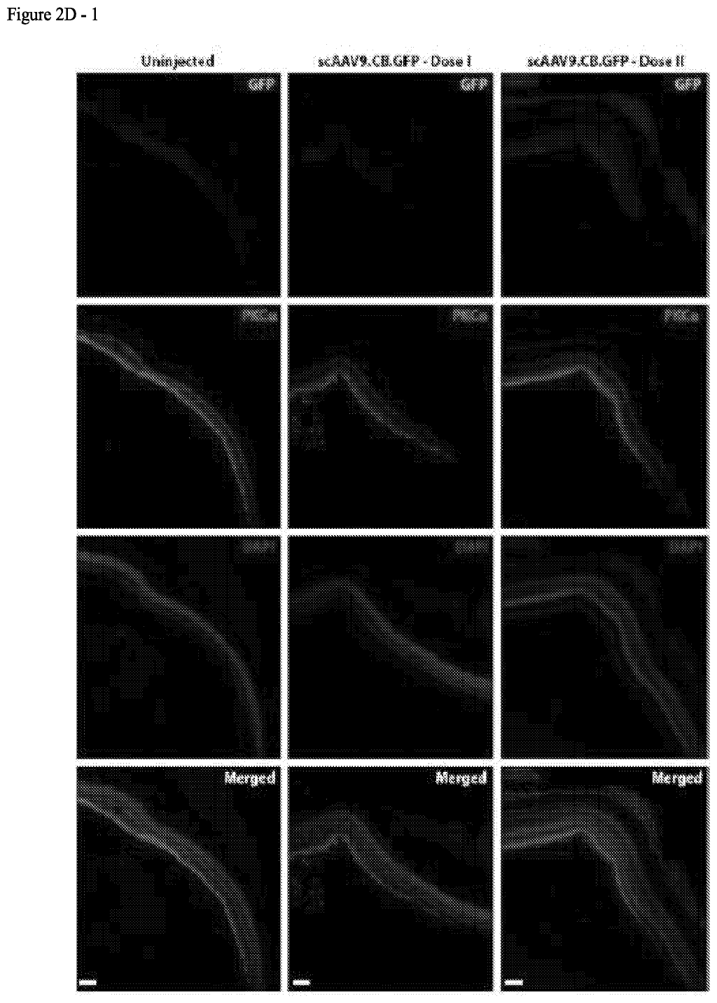

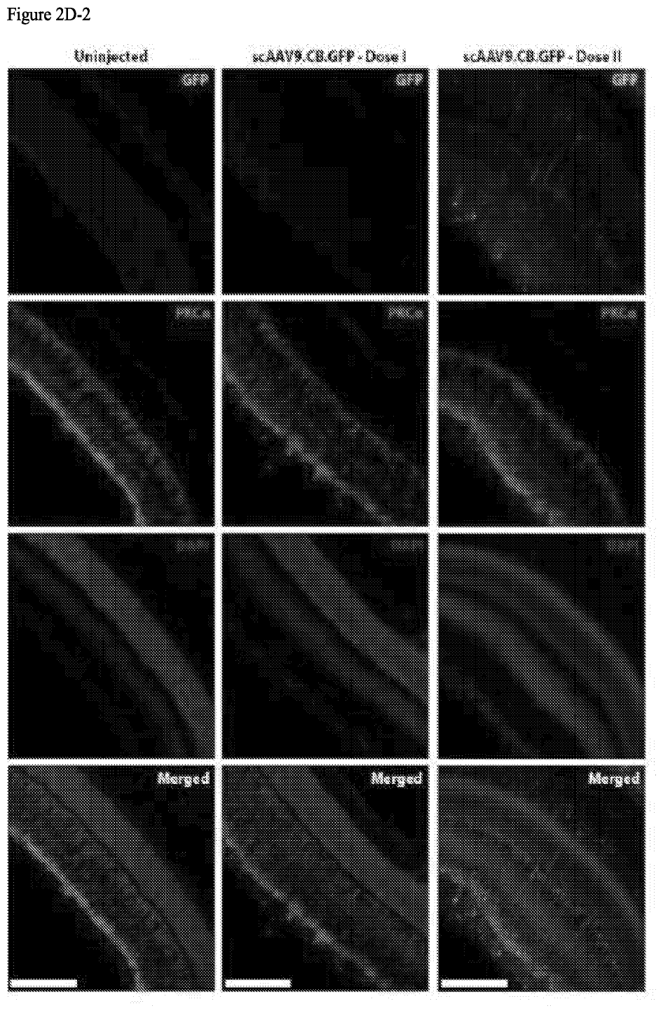

To examine the retinal expression of the transgene, an immunohistochemistry analysis was performed to visualize GFP protein. scAAV9.CB.GFP-injected mice were used. The retina tissue was stained for GFP (top row), PKCα (middle rows) which is a marker for rod bipolar cells and Draq5 (bottom rows) which provides nuclear counterstaining. As demonstrated in FIG. 2A, the rod bipolar cells expressed transgene GFP after ICV administration of scAAV9.CB.GFP.

Pax6 is a marker for amacrine/progenitor cells. As shown in FIG. 2B, ICV administration of scAAV9.CB.GFP at a dose of 5e10 vg resulted in expression of GFP in the rod bipolar cells (see middle column) and in the amacrine/progenitor cells (see left and right columns). As shown in FIG. 2C, transduction of rod bipolar cells (middle and right columns) and amacrine/progenitor cells (right column) was also observed after ICV administration of scAAV9.CB.GFP at a dose of 5e10 vg. FIG. 2D provides a composite of all tissues after ICV administration of scAAV9.CB.GFP.

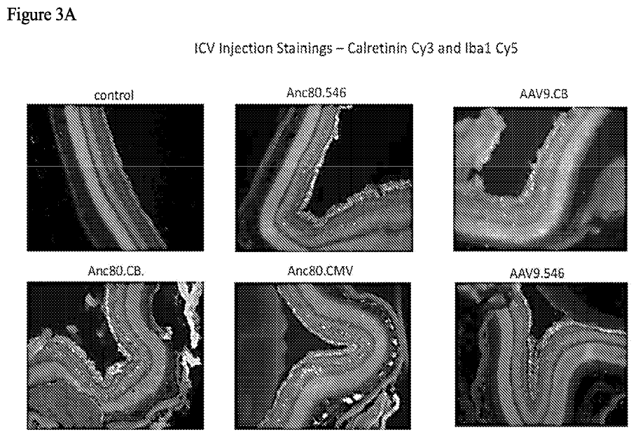

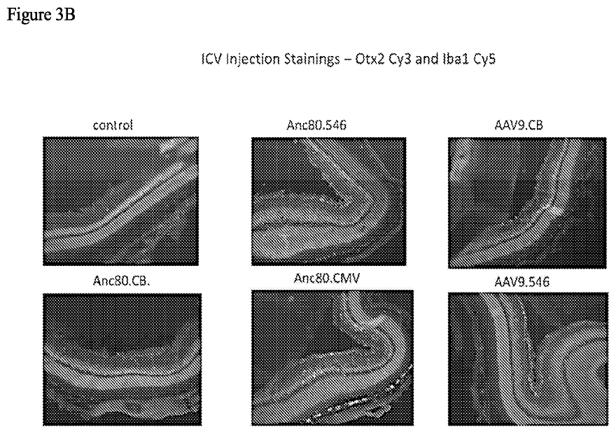

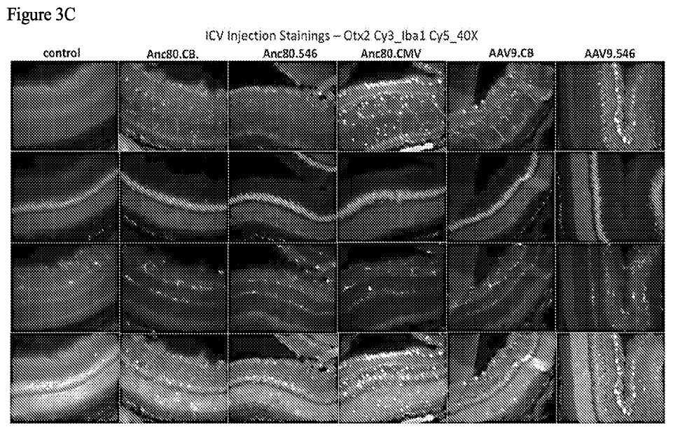

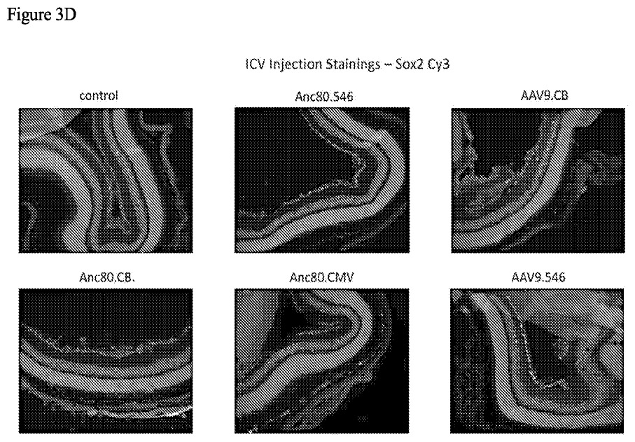

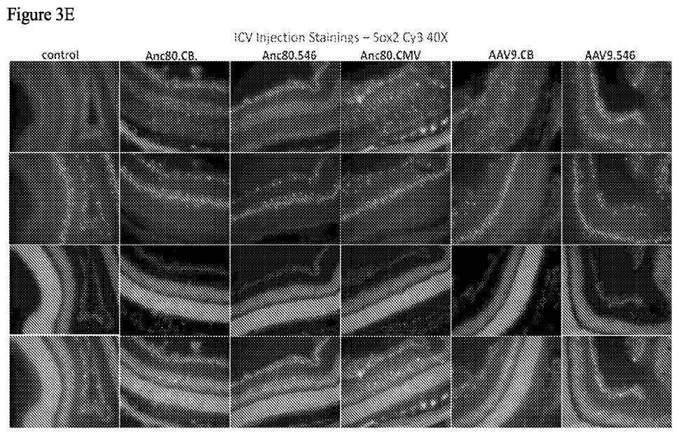

Calretinin (Cy3) is a marker for horizontal cells of the retina (red stain) and Iba1 (violet stain) is a marker for microglia cells of the retina. As shown in FIG. 3A, ICV administration of AAV9.CB.GFP, scAnc80.CB.GFP, scAnc80.CB.GFP and scAnc80.CMV delivered the transgene to microglia cells and the horizontal cells. Gene therapy vectors comprising the P546 promoter delivered the transgene at a lower rate than the CMV and CB promoter.

Otx2 is a nuclear marker for all bipolar cells (green stain) and Iba1 (red stain) is a marker for microglia cells of the retina. As shown in FIG. 3B, ICV administration of scAnc80.P546.GFP, AAV9.CB.GFP, scAnc80.CB.GFP, scAnc80.CMV and AAV9.P546.GFP delivered the transgene to bipolar cells and the microglia cells. A composite of Otx2 (red bipolar nuclei) and Iba1 (violet) staining is provided in FIG. 3C—

Sox2 is a maker for Mueller glia cells of the retina (green stain). These cells are involved in CLN3 disease. As shown in FIG. 3D, ICV administration of AAV9.CB.GFP, scAnc80.CB.GFP, scAnc80.CMV.GFP, and AAV9.P546.GFP delivered the transgene to the Mueller glia cells. Gene therapy vectors comprising the Anc80 vectors delivered the transgene at a lower rate than the AAV9 vector. A composite of the Sox2 staining is provided in FIG. 3E.

This experiment demonstrates that ICV administration of AAV resulted in delivery of the GFP transgene to rod biopolar cells and amacrine/progenitor cells within the retina. It is surprising that ICV delivery was very efficient in delivering transgene to the retinal cells.

### Example 3

Transduction of Mouse Retina after Sub-Retinal and Intrathecal Delivery

The mouse was anesthetized with isoflurane or Xylazene/Ketamine mix following standard procedures. A drop of Tropicamide was applied to dilate the pupil. A 4.0 suture was used to hold the eye forward by forming a small loop which is delicately wrapped around the eye, reducing movement for incision and injection methods. For sub-retinal injections, a small scleral incision was made, at or posterior to the equator with 30 G needle. Virus or vehicle was delivered sub-retinally via the incision using a fine glass pipette attached by tubing to a Hamilton syringe or via 30 G needle and Hamilton syringe. If needed, the suturing was performed using 10.0 sutures. Before and after the injection, ophthaine and vetropolycin were applied topically, mice were allowed to recover via standard of care (heated cage for recovery, food on the bottom of cage, long sipper tube) and monitored until stable.

scAAV9.CMV.GFP or scAnc80.CMV.GFP was administered to mice (1 to 5 months of age) via one sub-retinal injection and expression was monitored at various time points over a course of two months. The AAV and Anc80 were administered at a dose of ranging from 9×10⁹ and 3.2×10¹⁰ vg formulated PBS/F68.

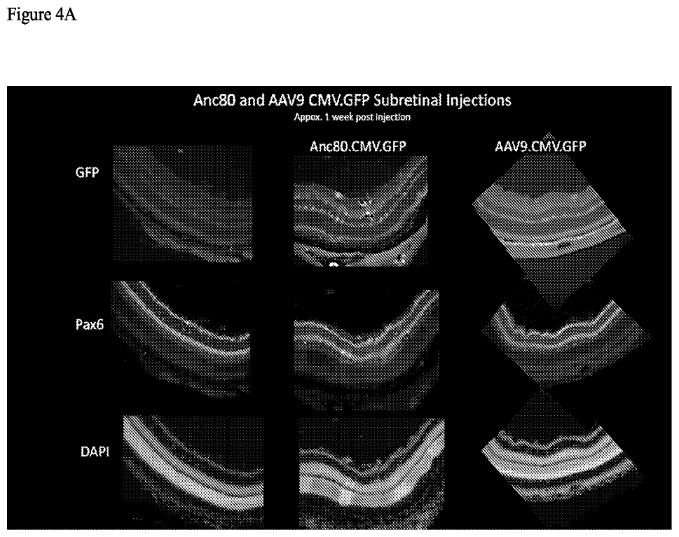

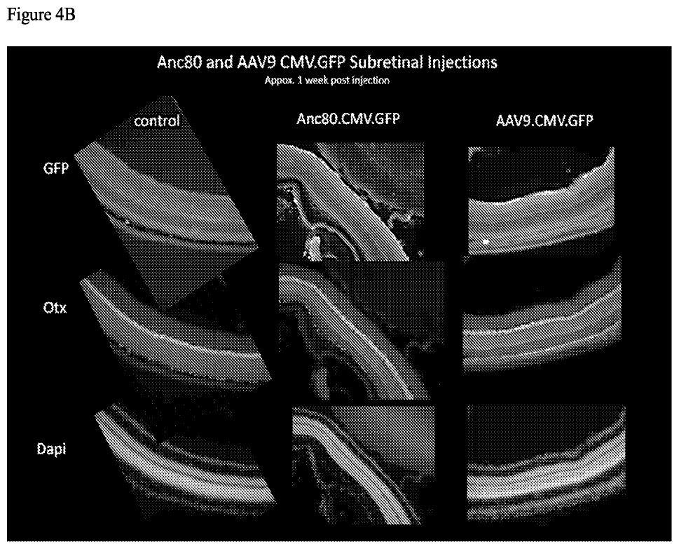

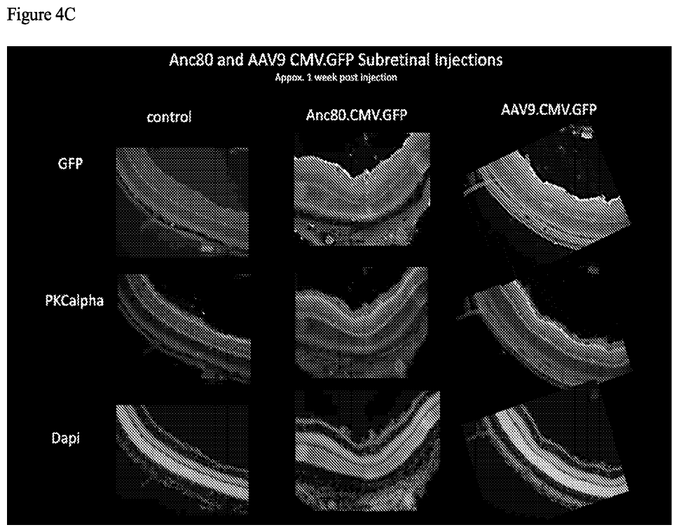

To examine the retinal expression of the transgene, an immunoshostochemistry analysis was used to visualize GFP protein. scAAV9.CMV.GFP or scAnc80.CMV.GFP-injected mice were used. The retina tissue was stained for GFP (top row), Pax6 (middle rows) which is a marker or amacrine/progenitor cells and DAPI (bottom rows) which provides nuclear counterstaining. As demonstrated in FIG. 4A, the amacrine/progenitors cells expressed transgene GFP after sub-retinal injection of scAAV9.CMV.GFP or scAnc80.CMV.GFP approximately one week after injection.

Otx2 is a nuclear marker for all retinal bipolar cells. Retina tissue was also stained for GFP (top row), Otx2 (middle rows) and DAPI (bottom rows) which provides nuclear counterstaining. As demonstrated in FIG. 4B, the bipolar cells expressed transgene GFP after sub-retinal injection scAAV9.CMV.GFP or scAnc80.CMV.GFP approximately one week after injection.

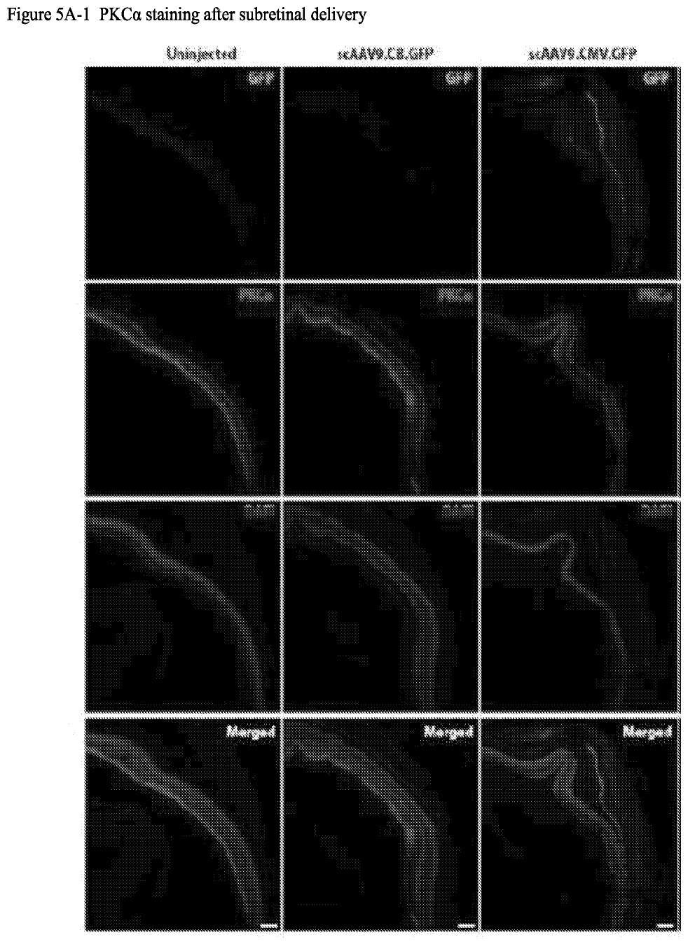

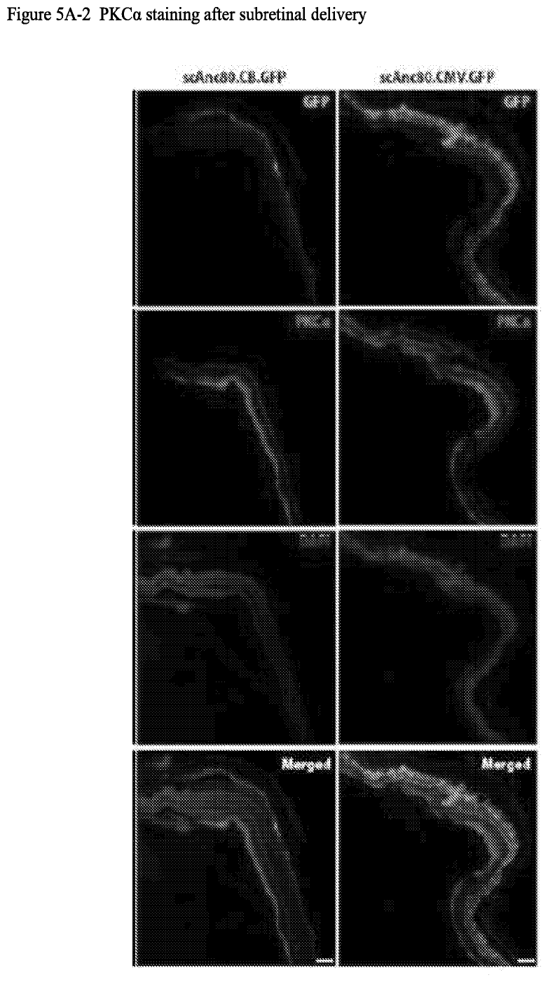

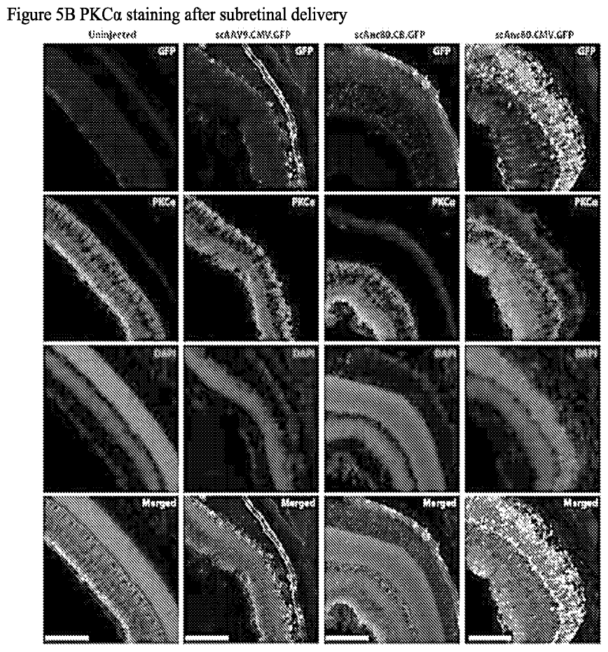

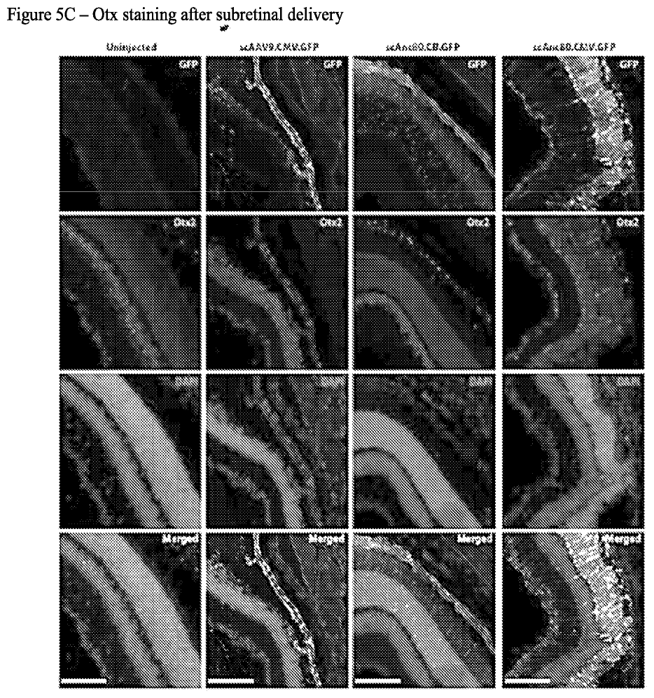

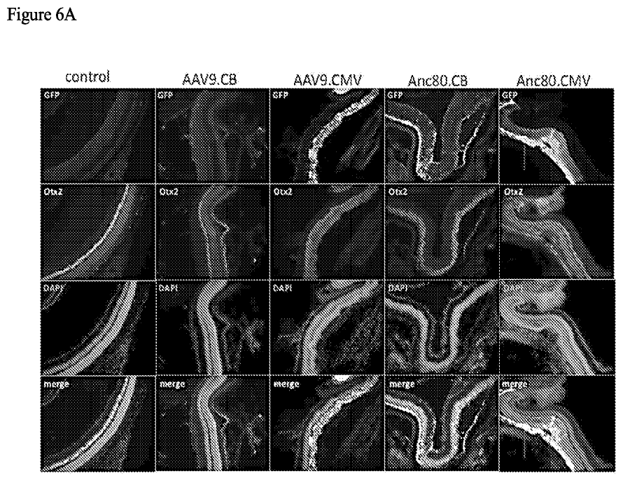

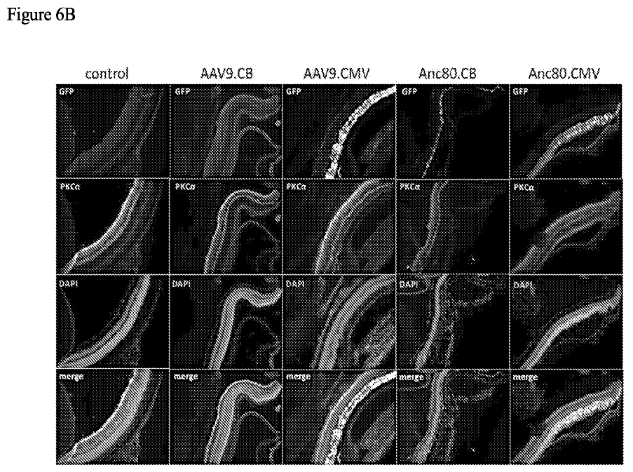

The retina tissue was also stained for GFP (top row), PKCα (middle rows) which is a marker for rod bipolar cells and DAPI (bottom rows) which provides nuclear counterstaining. As demonstrated in FIG. 4C, the rod bipolar cells expressed transgene GFP after subretinal injection or scAAV9.CMV.GFP or scAnc80.CMV.GFP approximately one week after injection. FIG. 5A-C provides a composite of all tissues after sub-retinal delivery of scAAV9.CB.GFP, scAAV9.CMV.GFP, scAnc80.CB.GFP or scAnc80.CMV.GFP. FIG. 5A shows the staining at a lower magnification for all 4 vectors tested. FIG. 5B shows staining at a high magnification, while FIG. 5C shows staining a high magnification but with a different co-stain. The transgene expression in the bipolar cells (Otx and PKCα staining) was detectable 4 weeks after subretinal injection of the following vectors: scAAV9.CB.GFP, scAnc80CMV.GFP, scAnc80.CB.GFP or scAnc80.CMV.GFP. FIG. 6 provides a composite of all tissues after sub-retinal delivery at low magnification for all 4 vectors tested using two different co-stains.

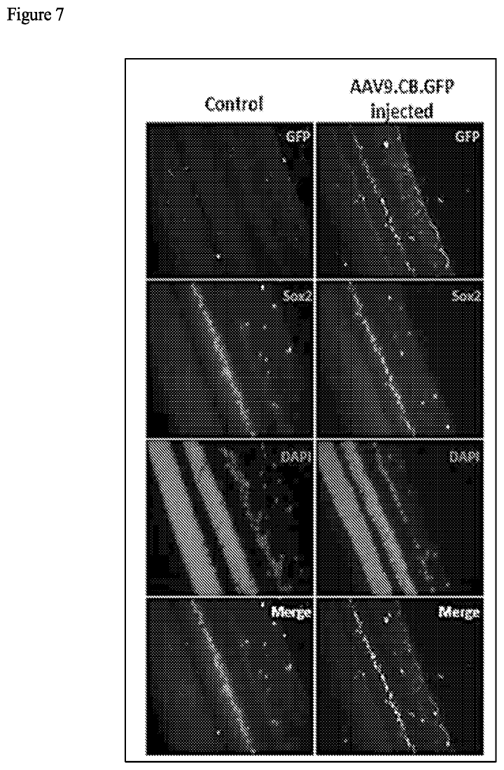

A ten-year old non-human primate was intrathecally injected with 1e14 vg of AAV9.CB.GFP. The retinas were counterstained with Sox2, a Mueller glia cell marker. FIG. 7 demonstrates marked co-localization of GFP and Sox2 in the primates injected with AAV9.CB.GFP.

### Example 4

Transduction of Mouse Retina after Intravitreal Delivery

The mouse was is anesthetized for the intravitreal injection as described above. A small incision was made between the limbus and sclera with 30 G needle. Virus or vehicle is delivered into vitreous space via the incision using a fine glass pipette attached by tubing to a Hamilton syringe or via 30 G needle and Hamilton syringe. Before and after the injection, ophthaine and vetropolycin are applied topically, mice are allowed to recover via standard of care (heated cage for recovery, food on the bottom of cage, long sipper tube) and monitored until stable

scAAV9.GFP or scANC80.GFP under the control of CB (promoter 1) or P546 (promoter 2) were administered to mice (1-5 months old) via one intravitreal injection and expression was monitored at various time points over a course of two months. The AAV and Anc80 were administered at a dose of 2×10¹⁰ vp formulated PBS/F68. The following table provides a guide for the cell markers used in this study.

| Retinal Cell Type | Retinal Layer | Antibody |
|---|---|---|
| Bipolar Cells (All) | Inner Nuclear Layer (INL) | Otx2 |
| Bipolar Cells (Rod) | Inner Nuclear Layer | PKCα |
| Mueller Glia | All w/nuclei in the INL | Sox2 |
| Photoreceptors (Rod) | Outer Nuclear Layer | Rhodopsin |
| Amacrine | Inner Nuclear Layer | Pax6 |
| Horizontal | Inner Nuclear Layer | Calretinin |
| Microglia | Inner Nuclear Layer | lba1 |

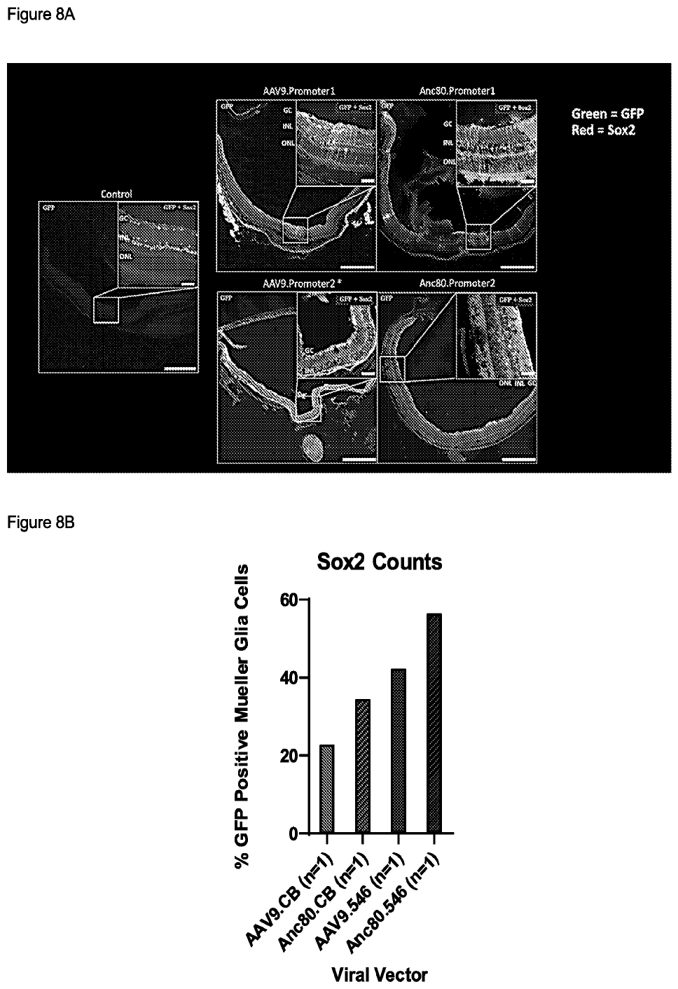

As shown in FIG. 8, intravitreal injections of any of the vectors tested resulted in GFP expression targeting the inner nuclear layer (INL). The retinal tissue was stained with GFP and Sox2, which is a marker that is specific for Mueller Cells within the INL. As shown in FIG. 8A, AAV9 and Anc80 are candidate vectors for targeting the inner and outer layer of the retina. FIG. 8B, provides the quantitative measurement of GPF positive Mueller glia (Sox2 staining). This measurement demonstrates that the all vectors tested transduced the Mueller glia with higher numbers of GFP positive cells obtained with P546 promoter than CBA promoter. This difference may be due to the difference in expression of these promoters within the Mueller glial cells. None the less, with comparable number of cells transduced between AAV9 and Anc80 vector, AAV9 has the added benefit as this vector can be utilized for CNS expression as well as retinal expression. However, Anc80 vector transduced a greater number of Mueller glia compared to AAV9.

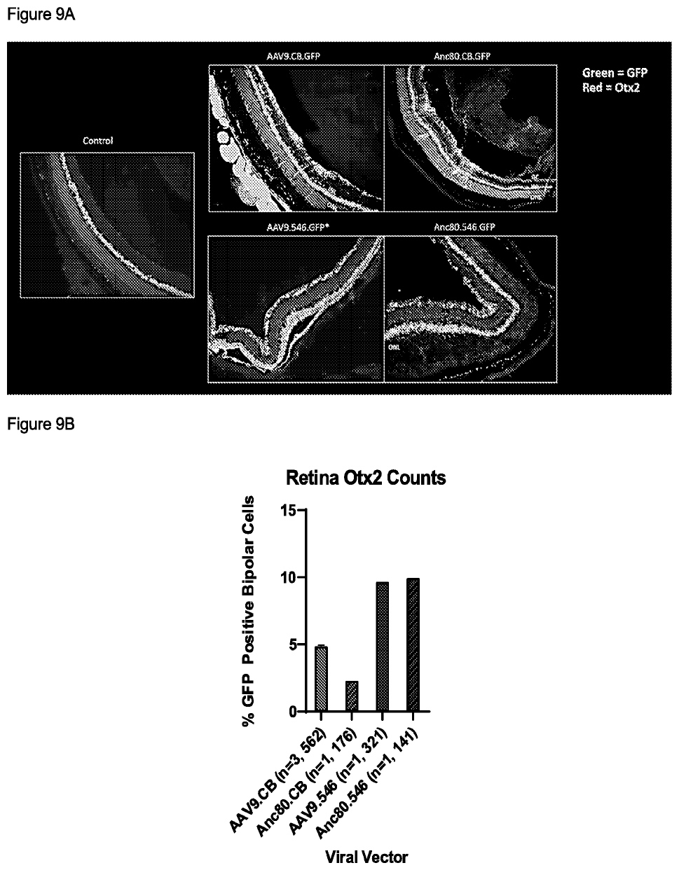

The retinal tissue was also stained with Otx2, which is a bipolar cell specific marker. As shown in FIG. 9A, AAV9.GFP and Anc80.GFP under the control of either CB or P546 transduced bipolar cells when delivered via intravitreal injection. Quantitative measurement show low percentages of GFP positive bipolar cell with all vectors tested (FIG. 9B). AAV9 and Anc80 vectors showed similar transduction rates in bipolar cells; while the P546 promoter allowed for better GFP expression in bipolar cells compared to the CB promoter.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein may be employed. It is intended that the following claims define the scope of the disclosure and that methods and structures within the scope of these claims and their equivalents be covered thereby.

All documents referred to in this application are hereby incorporated by reference in their entirety.

### REFERENCES

Arsov, T., Smith, K. R., Damiano, J., Franceschetti, S., Canafoglia, L., Bromhead, C. J., . . . Berkovic, S. F. (2011). Kufs disease, the major adult form of neuronal ceroid lipofuscinosis, caused by mutations in CLN6. American Journal of Human Genetics, 88(5), 566-573. https://doi.org/10.1016/j.ajhg.2011.04.004
Bras, J., Verloes, A., Schneider, S. A., Mole, S. E., & Guerreiro, R. J. (2012). Mutation of the parkinsonism gene ATP13A2 causes neuronal ceroid-lipofuscinosis. Human Molecular Genetics, 21(12), 2646-2650. https://doi.org/10.1093/hmg/dds089
Canafoglia, L., Morbin, M., Scaioli, V., Pareyson, D., D'Incerti, L., Fugnanesi, V., . . . Franceschetti, S. (2014). Recurrent generalized seizures, visual loss, and palinopsia as phenotypic features of neuronal ceroid lipofuscinosis due to progranulin gene mutation. Epilepsia, 55(6), e56-e59. https://doi.org/10.1111/epi.12632
Cannelli, N., Cassandrini, D., Bertini, E., Striano, P., Fusco, L., Gaggero, R., . . . Santorelli, F. M. (2006). Novel mutations in CLN8 in Italian variant late infantile neuronal ceroid lipofuscinosis: another genetic hit in the Mediterranean. Neurogenetics, 7(2), 111-117. https://doi.org/10.1007/s10048-005-0024-y
Estrada-Cuzcano, A., Martin, S., Chamova, T., Synofzik, M., Timmann, D., Holemans, T., . . . Schüle, R. (2017). Loss-of-function mutations in the ATP13A2/PARKS gene cause complicated hereditary spastic paraplegia (SPG78). Brain: A Journal of Neurology, 140(2), 287-305. https://doi.org/10.1093/brain/aww307
Kniffin, C. (2016). OMIM Clinical Synopsis-%609055-CEROID LIPOFUSCINOSIS, NEURONAL, 9; CLN9. Retrieved Mar. 19, 2019, from http://omim.org/clinicalSynopsis/609055
Moen, M. N., Fjær, R., Hamdani, E. H., Laerdahl, J. K., Menchini, R. J., Vigeland, M. D., . . . Chaudhry, F. A. (2016). Pathogenic variants in KCTD7 perturb neuronal K⁺ fluxes and glutamine transport. Brain, 139(12), 3109-3120. https://doi.org/10.1093/brain/aww244
Mole, S. E., & Williams, R. E. (1993). Neuronal Ceroid-Lipofuscinoses. GeneReviews®. University of Washington, Seattle. Retrieved from http://www.ncbi.nlm.nih.gov/pubmed/20301601
Nosková, L., Stránecký, V., Hartmannová, H., Přistoupilová, A., Barešová, V., Ivánek, R., . . . Kmoch, S. (2011). Mutations in DNAJC5, encoding cysteine-string protein alpha, cause autosomal-dominant adult-onset neuronal ceroid lipofuscinosis. American Journal of Human Genetics, 89(2), 241-252. https://doi.org/10.1016/j.ajhg.2011.07.003
Roosing, S., van den Born, L. I., Sangermano, R., Banfi, S., Koenekoop, R. K., Zonneveld-Vrieling, M. N., . . . Hoyng, C. B. (2015). Mutations in MFSD8, Encoding a Lysosomal Membrane Protein, Are Associated with Nonsyndromic Autosomal Recessive Macular Dystrophy. Ophthalmology, 122(1), 170-179. https://doi.org/10.1016/j.ophtha.2014.07.040
Staropoli, J. F., Karaa, A., Lim, E. T., Kirby, A., Elbalalesy, N., Romansky, S. G., . . . Cotman, S. L. (2012). A homozygous mutation in KCTD7 links neuronal ceroid lipofuscinosis to the ubiquitin-proteasome system. American Journal of Human Genetics, 91(1), 202-208. https://doi.org/10.1016/j.ajhg.2012.05.023
Weizmann Institute of Science. (2019). Ceroid Lipofuscinosis, Neuronal, 10 disease: Malacards-Research Articles, Drugs, Genes, Clinical Trials. Retrieved Mar. 19, 2019, from https://www.malacards.org/card/ceroid_lipofuscinosis_neuronal_10_2
Xin, W., Mullen, T. E., Kiely, R., Min, J., Feng, X., Cao, Y., . . . Sims, K. (2010). CLN5 mutations are frequent in juvenile and late-onset non-Finnish patients with NCL. Neurology, 74(7), 565-571. https://doi.org/10.1212/WNL.0b013e3181cff70d

| CLN3 nucleotide sequence (SEQ ID NO: 1) | |
|---|---|
| atgggaggct gtgcaggctc gcggcggcgc ttttcggatt ccgaggggga ggagaccgtc | 60 |
| ccggagcccc ggctccctct gttggaccat cagggcgcgc attggaagaa cgcggtgggc | 120 |
| ttctggctgc tgggcctttg caacaacttc tcttatgtgg tgatgctgag tgccgcccac | 180 |
| gacatcctta gccacaagag gacatcggga aaccagagcc atgtggaccc aggcccaacg | 240 |
| ccgatccccc acaacagctc atcacgattt gactgcaact ctgtctctac ggctgctgtg | 300 |
| ctcctggcgg acatcctccc cacactcgtc atcaaattgt tggctcctct tggccttcac | 360 |
| ctgctgccct acagcccccg ggttctcgtc agtgggattt gtgctgctgg aagcttcgtc | 420 |
| ctggttgcct tttctcattc tgtggggacc agcctgtgtg gtgtggtctt cgctagcatc | 480 |
| tcatcaggcc ttggggaggt caccttcctc tccctcactg ccttctaccc cagggccgtg | 540 |
| atctcctggt ggtcctcagg gactggggga gctgggctgc tgggggccct gtcctacctg | 600 |
| ggcctcaccc aggccggcct ctcccctcag cagaccctgc tgtccatgct gggtatccct | 660 |
| gccctgctgc tggccagcta tttcttgttg ctcacatctc ctgaggccca ggaccctgga | 720 |
| ggggaagaag aagcagagag cgcagcccgg cagcccctca taagaaccga ggccccggag | 780 |
| tcgaagccag gctccagctc cagcctctcc cttcgggaaa ggtggacagt gttcaagggt | 840 |
| ctgctgtggt acattgttcc cttggtcgta gtttactttg ccgagtattt cattaaccag | 900 |
| ggactttttg aactcctctt tttctggaac acttccctga gtcacgctca gcaataccgc | 960 |
| tggtaccaga tgctgtacca ggctggcgtc tttgcctccc gctcttctct ccgctgctgt | 1020 |
| cgcatccgtt tcacctgggc cctggccctg ctgcagtgcc tcaacctggt gttcctgctg | 1080 |
| gcagacgtgt ggttcggctt tctgccaagc atctacctcg tcttcctgat cattctgtat | 1140 |
| gaggggctcc tgggaggcgc agcctacgtg aacaccttcc acaacatcgc cctggagacc | 1200 |
| agtgatgagc accgggagtt tgcaatggcg gccacctgca tctctgacac actggggatc | 1260 |
| tccctgtcgg ggctcctggc tttgcctctg catgacttcc tctgccagct ctcctga | 1317 |
| CLN3 amino acid sequence (SEQ ID NO: 2) | |
| Met Gly Gly Cys Ala Gly Ser Arg Arg Arg Phe Ser Asp Ser Glu Gly | |
| 1 5 10 15 | |
| Glu Glu Thr Val Pro Glu Pro Arg Leu Pro Leu Leu Asp His Gln Gly | |
| 20 25 30 | |
| Ala His Trp Lys Asn Ala Val Gly Phe Trp Leu Leu Gly Leu Cys Asn | |
| 35 40 45 | |
| Asn Phe Ser Tyr Val Val Met Leu Ser Ala Ala His Asp Ile Leu Ser | |
| 50 55 60 | |
| His Lys Arg Thr Ser Gly Asn Gln Ser His Val Asp Pro Gly Pro Thr | |
| 65 70 75 80 | |
| Pro Ile Pro His Asn Ser Ser Ser Arg Phe Asp Cys Asn Ser Val Ser | |
| 85 90 95 | |
| Thr Ala Ala Val Leu Leu Ala Asp Ile Leu Pro Thr Leu Val Ile Lys | |
| 100 105 110 | |
| Leu Leu Ala Pro Leu Gly Leu His Leu Leu Pro Tyr Ser Pro Arg Val | |
| 115 120 125 | |
| Leu Val Ser Gly Ile Cys Ala Ala Gly Ser Phe Val Leu Val Ala Phe | |
| 130 135 140 | |
| Ser His Ser Val Gly Thr Ser Leu Cys Gly Val Val Phe Ala Ser Ile | |
| 145 150 155 160 | |
| Ser Ser Gly Leu Gly Glu Val Thr Phe Leu Ser Leu Thr Ala Phe Tyr | |
| 165 170 175 | |
| Pro Arg Ala Val Be Ser Trp Trp Ser Ser Gly Thr Gly Gly Ala Gly | |
| 180 185 190 | |
| Leu Leu Gly Ala Leu Ser Tyr Leu Gly Leu Thr Gln Ala Gly Leu Ser | |
| 195 200 205 | |
| Pro Gln Gln Thr Leu Leu Ser Met Leu Gly Ile Pro Ala Leu Leu Leu | |
| 210 215 220 | |
| Ala Ser Tyr Phe Leu Leu Leu Thr Ser Pro Glu Ala Gln Asp Pro Gly | |
| 225 230 235 240 | |
| Gly Glu Glu Glu Ala Glu Ser Ala Ala Arg Gln Pro Leu Ile Arg Thr | |
| 245 250 255 | |
| Glu Ala Pro Glu Ser Lys Pro Gly Ser Ser Ser Ser Leu Ser Leu Arg | |
| 260 265 270 | |
| Glu Arg Trp Thr Val Phe Lys Gly Leu Leu Trp Tyr Ile Val Pro Leu | |
| 275 280 285 | |
| Val Val Val Tyr Phe Ala Glu Tyr Phe Ile Asn Gln Gly Leu Phe Glu | |
| 290 295 300 | |
| Leu Leu Phe Phe Trp Asn Thr Ser Leu Ser His Ala Gln Gln Tyr Arg | |
| 305 310 315 320 | |
| Trp Tyr Gln Met Leu Tyr Gln Ala Gly Val Phe Ala Ser Arg Ser Ser | |
| 325 330 335 | |
| Leu Arg Cys Cys Arg Ile Arg Phe Thr Trp Ala Leu Ala Leu Leu Gln | |
| 340 345 350 | |
| Cys Leu Asn Leu Val Phe Leu Leu Ala Asp Val Trp Phe Gly Phe Leu | |
| 355 360 365 | |
| Pro Ser Ile Tyr Leu Val Phe Leu Ile Ile Leu Tyr Glu Gly Leu Leu | |
| 370 375 380 | |
| Gly Gly Ala Ala Tyr Val Asn Thr Phe His Asn Ile Ala Leu Glu Thr | |
| 385 390 395 400 | |
| Ser Asp Glu His Arg Glu Phe Ala Met Ala Ala Thr Cys Ile Ser Asp | |
| 405 410 415 | |
| Thr Leu Gly Ile Ser Leu Ser Gly Leu Leu Ala Leu Pro Leu His Asp | |
| 420 425 430 | |
| Phe Leu Cys Gln Leu Ser | |
| 435 | |
| CLN6 nucleotide sequence (SEQ ID NO: 3) | |
| atggaggcga cgcggaggcg gcagcacctg ggagcgacgg gcggcccagg cgcgcagctg | 60 |
| ggcgcctcct tcctgcaggc caggcatggc tctgtgagcg ctgatgaggc tgcccgcacg | 120 |
| gctcccttcc acctcgacct ctggttctac ttcacactgc agaactgggt tctggacttt | 180 |
| gggcgtccca ttgccatgct ggtattccct ctcgagtggt ttccactcaa caagcccagt | 240 |
| gttggggact acttccacat ggcctacaac gtcatcacgc cctttctctt gctcaagctc | 300 |
| atcgagcggt ccccccgcac cctgccacgc tccatcacgt acgtgagcat catcatcttc | 360 |
| atcatgggtg ccagcatcca cctggtgggt gactctgtca accaccgcct gctcttcagt | 420 |
| ggctaccagc accacctgtc tgtccgtgag aaccccatca tcaagaatct caagccggag | 480 |
| acgctgatcg actcctttga gctgctctac tattatgatg agtacctggg tcactgcatg | 540 |
| tggtacatcc ccttcttcct catcctcttc atgtacttca gcggctgctt tactgcctct | 600 |
| aaagctgaga gcttgattcc agggcctgcc ctgctcctgg tggcacccag tggcctgtac | 660 |
| tactggtacc tggtcaccga gggccagatc ttcatcctct tcatcttcac cttcttcgcc | 720 |
| atgctggccc tcgtcctgca ccagaagcgc aagcgcctct tcctggacag caacggcctc | 780 |
| ttcctcttct cctccttcgc actgaccctc ttgcttgtgg cgctctgggt cgcctggctg | 840 |
| tggaatgacc ctgttctcag gaagaagtac ccgggtgtca tctacgtccc tgagccctgg | 900 |
| gctttctaca cccttcacgt cagcagtcgg cactga | 936 |
| CLN6 amino acid sequence (SEO ID NO: 4): | |
| Met Glu Ala Thr Arg Arg Arg Gln His Leu Gly Ala Thr Gly Gly Pro | |
| 1 5 10 15 | |
| Gly Ala Gln Leu Gly Ala Ser Phe Leu Gln Ala Arg His Gly Ser Val | |
| 20 25 30 | |
| Ser Ala Asp Glu Ala Ala Arg Thr Ala Pro Phe His Leu Asp Leu Trp | |
| 35 40 45 | |
| Phe Tyr Phe Thr Leu Gln Asn Trp Val Leu Asp Phe Gly Arg Pro Ile | |
| 50 55 60 | |
| Ala Met Leu Val Phe Pro Leu Glu Trp Phe Pro Leu Asn Lys Pro Ser | |
| 65 70 75 80 | |
| Val Gly Asp Tyr Phe His Met Ala Tyr Asn Val Ile Thr Pro Phe Leu | |
| 85 90 95 | |
| Leu Leu Lys Leu Ile Glu Arg Ser Pro Arg Thr Leu Pro Arg Ser Ile | |
| 100 105 110 | |
| Thr Tyr Val Ser Ile Ile Ile Phe Be Met Gly Ala Ser Ile His Leu | |
| 115 120 125 | |
| Val Gly Asp Ser Val Asn His Arg Leu Leu Phe Ser Gly Tyr Gln His | |
| 130 135 140 | |
| His Leu Ser Val Arg Glu Asn Pro Ile Ile Lys Asn Leu Lys Pro Glu | |
| 145 150 155 160 | |
| Thr Leu Ile Asp Ser Phe Glu Leu Leu Tyr Tyr Tyr Asp Glu Tyr Leu | |
| 165 170 175 | |
| Gly His Cys Met Trp Tyr Ile Pro Phe Phe Leu Ile Leu Phe Met Tyr | |
| 180 185 190 | |
| Phe Ser Gly Cys Phe Thr Ala Ser Lys Ala Glu Ser Leu Ile Pro Gly | |
| 195 200 205 | |
| Pro Ala Leu Leu Leu Val Ala Pro Ser Gly Leu Tyr Tyr Trp Tyr Leu | |
| 210 215 220 | |
| Val Thr Glu Gly Gln Ile Phe Ile Leu Phe Ile Phe Thr Phe Phe Ala | |
| 225 230 235 240 | |
| Met Leu Ala Leu Val Leu His Gln Lys Arg Lys Arg Leu Phe Leu Asp | |
| 245 250 255 | |
| Ser Asn Gly Leu Phe Leu Phe Ser Ser Phe Ala Leu Thr Leu Leu Leu | |
| 260 265 270 | |
| Val Ala Leu Trp Val Ala Trp Leu Trp Asn Asp Pro Val Leu Arg Lys | |
| 275 280 285 | |
| Lys Tyr Pro Gly Val Ile Tyr Val Pro Glu Pro Trp Ala Phe Tyr Thr | |
| 290 295 300 | |
| Leu His Val Ser Ser Arg His | |
| 305 310 | |
| CLN8 nucleotide sequence (SEO ID NO: 5) | |
| atgaatcctg cgagcgatgg gggcacatca gagagcattt ttgacctgga ctatgcatcc | 60 |
| tgggggatcc gctccacgct gatggtcgct ggctttgtct tctacttggg cgtctttgtg | 120 |
| gtctgccacc agctgtcctc ttccctgaat gccacttacc gttctttggt ggccagagag | 180 |
| aaggtcttct gggacctggc ggccacgcgt gcagtctttg gtgttcagag cacagccgca | 240 |
| ggcctgtggg ctctgctggg ggaccctgtg ctgcatgccg acaaggcgcg tggccagcag | 300 |
| aactggtgct ggtttcacat cacgacagca acgggattct tttgctttga aaatgttgca | 360 |
| gtccacctgt ccaacttgat cttccggaca tttgacttgt ttctggttat ccaccatctc | 420 |
| tttgcctttc ttgggtttct tggctgcttg gtcaatctcc aagctggcca ctatctagct | 480 |
| atgaccacgt tgctcctgga gatgagcacg ccctttacct gcgtttcctg gatgctctta | 540 |
| aaggcgggct ggtccgagtc tctgttttgg aagctcaacc agtggctgat gattcacatg | 600 |
| tttcactgcc gcatggttct aacctaccac atgtggtggg tgtgtttctg gcactgggac | 660 |
| ggcctggtca gcagcctgta tctgcctcat ttgacactgt tccttgtcgg actggctctg | 720 |
| cttacgctaa tcattaatcc atattggacc cataagaaga ctcagcagct tctcaatccg | 780 |
| gtggactgga acttcgcaca gccagaagcc aagagcaggc cagaaggcaa cgggcagctg | 840 |
| ctgcggaaga agaggccata g | 861 |

```
CLN8 amino acid sequence (SEO ID NO: 6)
Met Asn Pro Ala Ser Asp Gly Gly Thr Ser Glu Ser Ile Phe Asp Leu
1 5 10 15
Asp Tyr Ala Ser Trp Gly Ile Arg Ser Thr Leu Met Val Ala Gly Phe
            20 25 30
Val Phe Tyr Leu Gly Val Phe Val Val Cys His Gln Leu Ser Ser Ser
          35 40 45
Leu Asn Ala Thr Tyr Arg Ser Leu Val Ala Arg Glu Lys Val Phe Trp
    50 55 60
Asp Leu Ala Ala Thr Arg Ala Val Phe Gly Val Gln Ser Thr Ala Ala
65 70 75 80
Gly Leu Trp Ala Leu Leu Gly Asp Pro Val Leu His Ala Asp Lys Ala
                  85 90 95
Arg Gly Gln Gln Asn Trp Cys Trp Phe His Ile Thr Thr Ala Thr Gly
            100 105 110
Phe Phe Cys Phe Glu Asn Val Ala Val His Leu Ser Asn Leu Ile Phe
        115 120 125
Arg Thr Phe Asp Leu Phe Leu Val Ile His His Leu Phe Ala Phe Leu
    130 135 140
Gly Phe Leu Gly Cys Leu Val Asn Leu Gln Ala Gly His Tyr Leu Ala
145 150 155 160
Met Thr Thr Leu Leu Leu Glu Met Ser Thr Pro Phe Thr Cys Val Ser
                165 170 175
Trp Met Leu Leu Lys Ala Gly Trp Ser Glu Ser Leu Phe Trp Lys Leu
            180 185 190
Asn Gln Trp Leu Met Ile His Met Phe His Cys Arg Met Val Leu Thr
        195 200 205
Tyr His Met Trp Trp Val Cys Phe Trp His Trp Asp Gly Leu Val Ser
    210 215 220
Ser Leu Tyr Leu Pro His Leu Thr Leu Phe Leu Val Gly Leu Ala Leu
225 230 235 240
Leu Thr Leu Ile Ile Asn Pro Tyr Trp Thr His Lys Lys Thr Gln Gln
                  245 250 255
Leu Leu Asn Pro Val Asp Trp Asn Phe Ala Gln Pro Glu Ala Lys Ser
            260 265 270
Arg Pro Glu Gly Asn Gly Gln Leu Leu Arg Lys Lys Arg Pro
        275 280 285
CB promoter (SEQ ID NO: 7)
ccacgttctg cttcactctc cccatctccc ccccctcccc acccccaatt ttgtatttat
ttatttttta attattttgt gcagcgatgg gggcgggggg gggggggggg cgcgcgccag
gcggggcggg gcggggcgag gggcggggcg gggcgaggcg gagaggtgcg gcggcagcca
atcagagcgg cgcgctccga aagtttcctt ttatggcgag gcggcggcgg cggcggccct
ataaaaagcg aagcgcgcgg cgggcgggag
CMV promoter (SEQ ID NO: 8)
cgttacataa cttacggtaa atggcccgcc tggctgaccg cccaacgacc cccgcccatt
gacgtcaata atgacgtatg ttcccatagt aacgccaata gggactttcc attgacgtca
atgggtggag tatttacggt aaactgccca cttggcagta catcaagtgt atcatatgcc
aagtacgccc cctattgacg tcaatgacgg taaatggccc gcctggcatt atgcccagta
catgacctta tgggactttc ctacttggca gtacatctac
P546 Promoter (SEQ ID NO: 9)
gaacaacgccaggctcctcaacaggcaactttgctacttctacagaaaatgataataaag
aaatgctggtgaagtcaaatgcttatcacaatggtgaactactcagcagggaggctctaa
taggcgccaagagcctagacttccttaagcgccagagtccacaagggcccagttaatcct
caacattcaaatgctgcccacaaaaccagcccctctgtgccctagccgcctcttttttcc
aagtgacagtagaactccaccaatccgcagctgaatggggtccgcctcttttccctgcct
aaacagacaggaactcctgccaattgagggcgtcaccgctaaggctccgccccagcctgg
gctccacaaccaatgaagggtaatctcgacaaagagcaaggggtggggcgcgggcgcgca
ggtgcagcagcacacaggctggtcgggagggcggggcgcgacgtctgccgtgcggggtcc
cggcatcggttgcgcgcgcgctccctcctctcggagagagggctgtggtaaaacccgtcc
ggaaaa
```

What is claimed:

1. A method of delivering a transgene to a retinal cell in a subject comprising administering a gene therapy vector encoding the transgene, wherein the gene therapy vector is administered to the subject using local intravenous delivery, sub-retinal delivery, intravitreous delivery or intrathecal delivery.

2. The method of claim 1 wherein the retinal cell is a bipolar cell, rod photoreceptor cell, cone photoreceptor cell, ganglion cell, Mueller glia cell, microglia cell, horizontal cell or amacrine cell.

3. A method of treating visual impairment or a vision-related disorder in a subject comprising administering a gene therapy vector encoding a transgene to the subject, wherein the gene therapy vector is administered using local intravenous delivery, sub-retinal delivery, intravitreous delivery or intrathecal delivery.

4. The method of claim 3 wherein the vision-related disorder is Batten disease, congenital cataracts, congenital glaucoma, retinal degeneration, optic atrophy, eye malformations. Strabismus, ocular misalignment, glaucoma, wet age-related macular degeneration, dry age-related macular degeneration, retinitis pigmentosa, choroideremia, Leber congenital amaurosis, Leber's hereditary optic neuropathy, early onset retinal dystrophy, achromatopsia, x-linked retinoschisis, Usher Syndrome 1B, neovascular age-related macular degeneration, Stargardt's macular degeneration, diabetic macular degeneration, or diabetic macular edema.

5. The method of claim 3 or 4 wherein the vision-related disorder is Batten disease or the visual impairment is a symptom of Batten disease.

6. The method of claim 5 wherein the Batten disease is CLN1 disease, CLN2 disease, CLN3 disease, CLN4 disease, CLN5 disease, CLN6 disease or CLN8 disease.

7. The method of any one of claims 1-6 wherein the transgene encodes RPE65, RPGR, ORF15, CNGA3, CMH, ND4, PDE6B, ChR2, MERTK, hRS1, hMYOJA, hABCA4, CD59, anti-hVEGF antibody, endostatin-angiostatin, sFLT01, or sFLT-1.

8. The method of any one of claims 1-7 wherein the transgene is a miRNA, siRNA against RTP801, siRNA against VEGFR-1, siRNA against VEGF, or siRNA against ADRB2.

9. The method of any one of claims 1-8 wherein the transgene encodes a CLN polypeptide.

10. The method of claim 9 wherein the CLN polypeptide is CLN1, CLN2, CLN3, CLN4, CLN5, CLN6 or CLN8.

11. A method of treating Batten disease in a subject comprising administering to the subject a gene therapy vector comprising a polynucleotide encoding a CLN polypeptide, wherein the gene therapy vector is administered using sub-retinal delivery, intravitreous delivery or intrathecal delivery.

12. The method of claim 11 wherein the Batten disease is CLN1 disease, CLN2 disease, CLN3 disease, CLN4 disease, CLN5 disease, CLN6 disease or CLN8 disease.

13. The method of any one of claim 11 or 12 wherein the transgene encodes a CLN polypeptide.

14. The method of claim 13 wherein the CLN polypeptide is CLN1, CLN2, CLN3, CLN4, CLN5, CLN6 or CLN8.

15. The method of any one of claims 11-14, wherein the treatment reduces or slows one or more symptoms of Batten Disease selected from:
(a) loss of vision;
(b) loss of brain volume;
(c) loss of cognitive function; and
(d) language delay;
as compared to an untreated Batten Disease patient.

16. The method of any one of claims 1-15 wherein the gene therapy vector is AAV1, AAV2, AAV3, AAV4, AAV5, AAV6, AAV7, AAV8, AAV9, AAVRH10, AAVRH74, AAV11, AAV12, AAV13, AAVTT or Anc80 or AAV7m8.

17. The method of any one of claims 1-15, wherein the AAV9 or Anc80 is administered using intrathecal delivery, and the method further comprises placing the subject in the Trendelenburg position after administering of the gene therapy vector.

18. A composition for delivering a transgene to a retinal cell in a subject, wherein the composition comprises a gene therapy vector encoding the transgene, wherein the composition is formulated for administering the gene therapy vector using local intravenous delivery, sub-retinal delivery, intravitreous delivery or intrathecal delivery.

19. The composition of claim 18 wherein the retinal cell is a bipolar cell, rod photoreceptor cell, cone photoreceptor cell, ganglion cell, Mueller glia cell, microglia cell, horizontal cell or amacrine cell.

20. A composition for treating visual impairment or a vision-related disorder in a subject, wherein the composition comprises a gene therapy vector encoding a transgene to the subject, wherein the composition is formulated for administering the gene therapy vector using local intravenous delivery, sub-retinal delivery, intravitreous delivery or intrathecal delivery.

21. The composition of claim 20 wherein the vision-related disorder is Batten disease, congenital cataracts, congenital glaucoma, retinal degeneration, optic atrophy, eye malformations. Strabismus, ocular misalignment, glaucoma, wet age-related macular degeneration, dry age-related macular degeneration, retinitis pigmentosa, choroideremia, Leber congenital amaurosis, Leber's hereditary optic neuropathy, early onset retinal dystrophy, achromatopsia, x-linked retinoschisis, Usher Syndrome 1B, neovascular age-related macular degeneration, Stargardt's macular degeneration, diabetic macular degeneration, or diabetic macular edema.

22. The composition of claim 20 or 21 wherein the vision-related disorder is Batten disease or the visual impairment is a symptom of Batten disease.

23. The composition of claim 22 wherein the Batten disease is CLN1 disease, CLN2 disease, CLN3 disease, CLN4 disease, CLN5 disease, CLN6 disease or CLN8 disease.

24. The composition of any one of claims 18-21 wherein the transgene encodes RPE65, RPGR, ORF15, CNGA3, CMH, ND4, PDE6B, ChR2, MERTK, hRS1, hMYOJA, hABCA4, CD59, anti-hVEGF antibody, endostatin-angiostatin, sFLT01, or sFLT-1.

25. The composition of any one of claims 18-21 wherein the transgene is a miRNA, siRNA against RTP801, siRNA against VEGFR-1, siRNA against VEGF, or siRNA against ADRB2.

26. The composition of any one of claims 18-23 wherein the transgene encodes a CLN polypeptide.

27. The composition of claim 26 wherein the CLN polypeptide is CLN1, CLN2, CLN3, CLN4, CLN5, CLN6 or CLN8.

28. A composition for treating Batten disease in a subject, wherein the composition comprises a gene therapy vector comprising a polynucleotide encoding a CLN polypeptide, wherein the composition is formulated for administering the gene therapy vector using sub-retinal delivery, intravitreous delivery or intrathecal delivery.

29. The composition of claim 28 wherein the Batten disease is CLN1 disease, CLN2 disease, CLN3 disease, CLN4 disease, CLN5 disease, CLN6 disease or CLN8 disease.

30. The composition of any one of claim 28 or 29 wherein the transgene encodes a CLN polypeptide.

31. The composition of claim 30 wherein the CLN polypeptide is CLN1, CLN2, CLN3, CLN4, CLN5, CLN6 or CLN8.

32. The composition of any one of claims 28-31, wherein the treatment reduces or slows one or more symptoms of Batten Disease selected from:
(a) loss of vision;
(b) loss of brain volume;
(c) loss of cognitive function; and
(d) language delay;
as compared to an untreated Batten Disease patient.

33. The composition of any one of claims 18-32 wherein the gene therapy vector is AAV1, AAV2, AAV3, AAV4, AAV5, AAV6, AAV7, AAV8, AAV9, AAVRH10, AAVRH74, AAV11, AAV12, AAV13, AAVTT or Anc80 or AAV7m8.

34. The composition of any one of claims 18-32, wherein the gene therapy vector is AAV9 or Anc80, and the composition is formulated for administering the gene therapy using intrathecal delivery, and the subject is placed in the Trendelenburg position after administering of the gene therapy vector.

35. Use of gene therapy for the preparation of a medicament for delivering a transgene to a retinal cell in a subject, wherein the medicament comprises a gene therapy vector encoding the transgene, and wherein the medicament is formulated for administering the gene therapy vector using local intravenous delivery, sub-retinal delivery, intravitreous delivery or intrathecal delivery.

36. The use of claim 35 wherein the retinal cell is a bipolar cell, rod photoreceptor cell, cone photoreceptor cell, ganglion cell, Mueller glia cell, microglia cell, horizontal cell or amacrine cell.

37. Use of a gene therapy vector the preparation of a medicament for treating visual impairment or a vision-related disorder in a subject, wherein the medicament comprises a gene therapy vector encoding a transgene, wherein the medicament is formulated for administering the gene therapy vector using local intravenous delivery, sub-retinal delivery, intravitreous delivery or intrathecal delivery.

38. The use of claim 37 wherein the vision-related disorder is Batten disease, congenital cataracts, congenital glaucoma, retinal degeneration, optic atrophy, eye malformations. Strabismus, ocular misalignment, glaucoma, wet age-related macular degeneration, dry age-related macular degeneration, retinitis pigmentosa, choroideremia, Leber congenital amaurosis, Leber's hereditary optic neuropathy, early onset retinal dystrophy, achromatopsia, x-linked retinoschisis, Usher Syndrome 1B, neovascular age-related macular degeneration, Stargardt's macular degeneration, diabetic macular degeneration, or diabetic macular edema.

39. The use of claim 37 or 38 wherein the vision-related disorder is Batten disease or the visual impairment is a symptom of Batten disease.

40. The use of claim 39 wherein the Batten disease is CLN1 disease, CLN2 disease, CLN3 disease, CLN4 disease, CLN5 disease, CLN6 disease or CLN8 disease.

41. The use of any one of claims 35-40 wherein the transgene encodes RPE65, RPGR, ORF15, CNGA3, CMH, ND4, PDE6B, ChR2, MERTK, hRS1, hMYOJA, hABCA4, CD59, anti-hVEGF antibody, endostatin-angiostatin, sFLT01, or sFLT-1.

42. The use of any one of claims 35-40 wherein the transgene is a miRNA, siRNA against RTP801, siRNA against VEGFR-1, siRNA against VEGF, or siRNA against ADRB2.

43. The use of any one of claims 35-40 wherein the transgene encodes a CLN polypeptide.

44. The use of claim 43 wherein the CLN polypeptide is CLN1, CLN2, CLN3, CLN4, CLN5, CLN6 or CLN8.

45. Use of a gene therapy vector for the preparation of a medicament for treating Batten disease in a subject, wherein the medicament comprises a gene therapy vector comprising a polynucleotide encoding a CLN polypeptide, and wherein the medicament is formulated for administering the gene therapy vector using sub-retinal delivery, intravitreous delivery or intrathecal delivery.

46. The use of claim 45 wherein the Batten disease is CLN1 disease, CLN2 disease, CLN3 disease, CLN4 disease, CLN5 disease, CLN6 disease or CLN8 disease.

47. The use of claim 45 or 46 wherein the transgene encodes a CLN polypeptide.

48. The use of claim 47 wherein the CLN polypeptide is CLN1, CLN2, CLN3, CLN4, CLN5, CLN6 or CLN8.

49. The use of any one of claims 35-48, wherein the treatment reduces or slows one or more symptoms of Batten Disease selected from:
(a) loss of vision;
(b) loss of brain volume;
(c) loss of cognitive function; and
(d) language delay;
as compared to an untreated Batten Disease patient.

50. The use of any one of claims 35-49 wherein the gene therapy vector is AAV1, AAV2, AAV3, AAV4, AAV5, AAV6, AAV7, AAV8, AAV9, AAVRH10, AAVRH74, AAV11, AAV12, AAV13, AAVTT or Anc80 or AAV7m8.

51. The use of any one of claims 35-49, wherein the gene therapy vector is AAV9 or Anc80, and the medicament is formulated for administering the gene therapy using intrathecal delivery, and the subject is placed in the Trendelenburg position after administering of the gene therapy vector.
